(19) United States
(12) Patent Application Publication
(10) Pub. No.: US 2022/0005606 A1
(43) Pub. Date: Jan. 6, 2022

(54) Methods of Determining the risk of Bovine Respiratory Disease in Pre-weaned Dairy Calves

(71) Applicant: The Regents of the University of California, Oakland, CA (US)

(72) Inventors: Sharif Aly, Tulare, CA (US); Gabriele Maier, Woodland, CA (US); Sasha Dubrovsky, Sacramento, CA (US)

(21) Appl. No.: 17/292,645

(22) PCT Filed: Nov. 13, 2019
(86) PCT No.: PCT/US2019/061197

§ 371 Date: May 10, 2021

Related U.S. Application Data
(60) Provisional application No. 62/767,031, filed on Nov. 14, 2018.

(51) Int. Cl. G16H 50/30

(52) U.S. Cl. CPC G16H 50/30

(57) ABSTRACT

Provided are methods of determining the risk of bovine respiratory disease (BRD) in pre-weaned dairy calves on a dairy farm. In certain aspects, the methods include extracting dairy farm management practice information from a dairy farm, wherein the dairy farm management practice information comprises one or more items of information for two or more dairy farm management practice categories selected from the group consisting of: basic herd risk profile, maternity pen management, colostrum management, milk feeding, vaccinations, and calf housing. In certain aspects, the methods further include assigning weighted risk scores to the items of dairy farm management practice information extracted, and determining a total risk score for each of the two or more dairy farm management practice categories based on the assigned weighted risk scores. Also provided are non-transitory computer-readable media and computer devices that find use, e.g., in practicing the methods of the present disclosure.

### CROSS-REFERENCE TO RELATED APPLICATIONS

This application claims the benefit of U.S. Provisional Patent Application No. 62/767,031 filed Nov. 14, 2018, which application is incorporated herein by reference in its entirety.

### STATEMENT OF GOVERNMENT SUPPORT

This invention was made with Government support under Grant No. CA_V_PHR_4041-H, awarded by the US Department of Agriculture (USDA). The Government has certain rights in the invention.

### INTRODUCTION

Bovine respiratory disease (BRD) is a significant disease of pre-weaned dairy calves. In 2010, respiratory disease in dairy heifers was reported as the cause in 22.5% of deaths before weaning (USDA, 2010), second only to diarrhea as the leading cause. Despite the availability of numerous vaccines and antimicrobial drugs for the prevention and treatment of the disease, morbidity and mortality have remained static over the years (USDA, 1991, 2012). BRD is a general term for respiratory disease in cattle caused by a range of factors, singly or in combination. A major cause of economic losses, BRD affects the lower respiratory tract/lungs (pneumonia) or upper respiratory tract (rhinitis, tracheitis, bronchitis). BRD is typically caused by a variety of pathogens, both viral (Bovine Respiratory Syncytial Virus (BRSV), Parainfluenza 3 (P13), Adenovirus, Bovine Viral Diarrhea Virus (BVDV), and Infectious Bovine Rhinotracheitis (IBR) and bacterial (Pasteurella multocida, Mannheimia haemolytica, Histophilus somni, Mycoplasma bovis). Parasitic (lungworm) and fungal (Aspergillus) agents are also pathogens. These pathogens interact with one another and the animal's immune system to produce disease, sub-optimum welfare conditions and loss of productivity. Bacterial pathogens may cause the acute syndrome by invading the bovine respiratory tract that has been compromised by viral infections, environmental conditions and/or other stress factors. Contributing to the disease complex is stress. Stressors include weaning, changes of feed, variation in ambient temperature and humidity, and weather.

### SUMMARY

Provided are methods of determining the risk of bovine respiratory disease (BRD) in pre-weaned dairy calves on a dairy farm. In certain aspects, the methods include extracting management practice information from a dairy farm, wherein the dairy farm management practice information comprises one or more items of information for two or more dairy farm management practice categories selected from the group consisting of: basic herd risk profile, maternity pen management, colostrum management, milk feeding, vaccinations, and calf housing. In certain aspects, the methods further include assigning weighted risk scores to the items of dairy farm management practice information extracted, and determining a total risk score for each of the two or more dairy farm management practice categories based on the assigned weighted risk scores. Also provided are non-transitory computer-readable media and computer devices that find use, e.g., in practicing the methods of the present disclosure.

### BRIEF DESCRIPTION OF THE FIGURES

FIG. 1 provides an overview of benchmarks for risk scores for risk of BRD. Darker numbers represent greater risk.

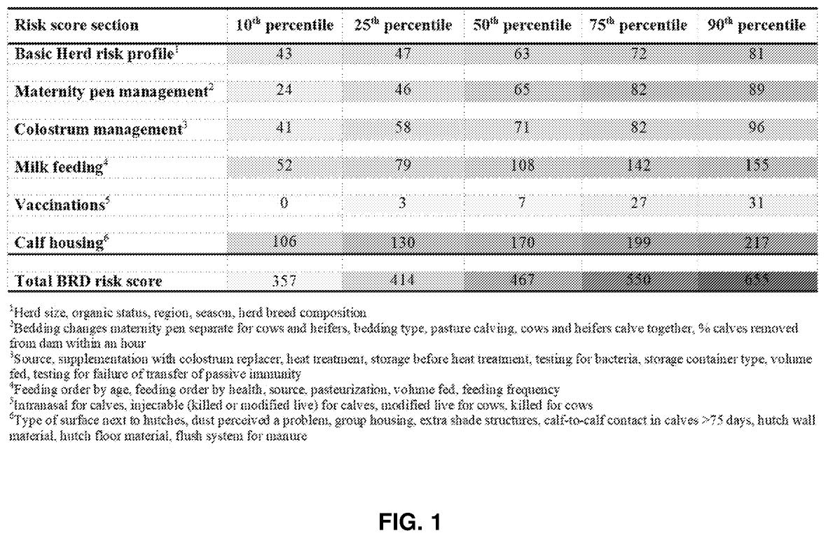

FIG. 2 provides an illustration of the BRD scoring system for pre-weaned dairy calves.

FIG. 3 shows how total scaled scores correlate with prevalence in the 100 study dairies of the BRD100 study.

### DETAILED DESCRIPTION

Provided are methods of determining the risk of bovine respiratory disease (BRD) in pre-weaned dairy calves on a dairy farm. In certain aspects, the methods include extracting management practice information from a dairy farm, wherein the dairy farm management practice information comprises one or more items of information for two or more dairy farm management practice categories selected from the group consisting of: basic herd risk profile, maternity pen management, colostrum management, milk feeding, vaccinations, and calf housing. In certain aspects, the methods further include assigning weighted risk scores to the items of dairy farm management practice information extracted, and determining a total risk score for each of the two or more dairy farm management practice categories based on the assigned weighted risk scores. Also provided are non-transitory computer-readable media and computer devices that find use, e.g., in practicing the methods of the present disclosure.

Before the methods, non-transitory computer-readable media and computer devices of the present disclosure are described in greater detail, it is to be understood that the methods, non-transitory computer-readable media and computer devices are not limited to particular embodiments described, as such may, of course, vary. It is also to be understood that the terminology used herein is for the purpose of describing particular embodiments only, and is not intended to be limiting, since the scope of the methods, non-transitory computer-readable media and computer devices will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, non-transitory computer-readable media and computer devices. The upper and lower limits of these smaller ranges may independently be included in the smaller ranges and are also encompassed within the methods, non-transitory computer-readable media and computer devices, subject to any specifically excluded limit in the stated range. Where the stated range includes one or both of the limits, ranges excluding either or both of those included limits are also included in the methods, non-transitory computer-readable media and computer devices.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, non-transitory computer-readable media and computer devices belong. Although any methods, non-transitory computer-readable media and computer devices similar or equivalent to those described herein can also be used in the practice or testing of the methods, non-transitory computer-readable media and computer devices, representative illustrative methods, non-transitory computer-readable media and computer devices are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, non-transitory computer-readable media and computer devices are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, non-transitory computer-readable media and computer devices, which are, for clarity, described in the context of separate embodiments, may also be provided in combination in a single embodiment. Conversely, various features of the methods, non-transitory computer-readable media and computer devices, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, non-transitory computer-readable media and computer devices and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Overview

The present disclosure provides methods, such as, computer-implemented methods as well as non-transitory readable media, computer programs, and the like for practicing these methods. These methods improve current management practices in dairy farms that rely heavily on antimicrobials without regard for the actual risk for development of BRD. The methods disclosed herein provide a quantitative assessment of BRD risk that permits, e.g., more judicious use of antimicrobials. In addition, these methods can be used for identifying the most effective management practices that mitigate the risk of BRD in pre-weaned dairy calves on a dairy farm leading to healthier calves and overall reduction in use of antimicrobials.

### Methods

As summarized above, the present disclosure provides methods of determining the risk of bovine respiratory disease (BRD) in pre-weaned dairy calves on a dairy farm. Details regarding the steps of the methods of the present disclosure will now be provided.

In certain aspects, the methods include extracting dairy farm management practice information from a dairy farm. In certain aspects, extracting dairy farm management practice information from a dairy farm may include administering a questionnaire comprising a plurality of questions regarding one or more items of information for two or more management practices in a dairy farm and recording the information provided in response to the questionnaire. In certain aspects, the two or more dairy farm management practice categories may be selected from the following management practice categories: basic herd risk profile, maternity pen management, colostrum management, milk feeding, vaccinations, and calf housing.

In certain aspects, the dairy farm management practice information comprises one or more items of basic herd risk profile information. In certain aspects, basic herd risk profile may include information regarding features inherent to the dairy herd, such as features that form the primary characteristics of the dairy herd. Expenditure of beyond reasonable cost, effort and time is usually required to change a basic herd profile. For example, interventions requiring beyond the available respective resources are required to change the basic herd profile. In certain aspects, the one or more items of basic herd risk profile information may include one or more of (e.g., 1, 2, 3, 4, or each of): herd size, breed distribution, organic herd status, and season. In certain aspects, the one or more items of basic herd risk profile information may include region. In certain aspects, region refers to geographical location of the dairy farm. In certain aspects, breed distribution refers to the make-up of the cattle population in the farm, e.g., presence or absence of different breeds of cows, relative population of the different breeds of cows, if present, and the like. In certain aspects, the one or more items of basic herd risk profile information may include information regarding one or more risk factors listed in Table 9, Section I. In certain aspects, two or more items of basic herd risk profile information may be extracted. In certain aspects, three or more items of basic herd risk profile information may be extracted. In certain aspects, four or more items of basic herd risk profile information may be extracted. In certain aspects, all five items of basic herd risk profile information may be extracted.

In certain aspects, the dairy farm management practice information may include one or more items of maternity pen management information. In certain aspects, maternity pen management include activities that contribute to exposure of pre-weaned calves to infectious agents during the perinatal period. In certain aspects, the one or more items of maternity pen management information includes one or more of (e.g., 1, 2, 3, 4, 5, or each of): frequency of bedding changes in the maternity pen for periparturient cows and heifers, individual or group pen calving for cows and heifers, whether cows and heifers calve together, the materials used for bedding, whether calves are born on pasture, and whether at least 95% of calves are removed from the dam within an hour of birth. In certain aspects, maternity pen management information may include information regarding one or more risk factors listed in Table 9, Section II. In certain aspects, two or more items of maternity pen management information may be extracted. In certain aspects, three or more items of maternity pen management information may be extracted. In certain aspects, four or more items of maternity pen management information may be extracted. In certain aspects, five or more items of maternity pen management information may be extracted. In certain aspects, all six items of maternity pen management information may be extracted.

In certain aspects, the dairy farm management practice information comprises one or more items of colostrum management information. In certain aspects, the one or more items of colostrum management information comprises one or more of (e.g., 1, 2, 3, 4, 5, 6, 7, 8, 9, 10, or each of): the source of colostrum, whether colostrum was supplemented with replacer, whether the colostrum was heat-treated before feeding, how long the colostrum was stored before heat-treatment, testing for bacterial content of colostrum, use of colostrum from first calf heifers, addition of a preservative, the type of storage container, testing for immunoglobulin content, whether calves received at least 2.84 L (3 quarts) of colostrum in the first 12 hours of life, and whether calves are tested for failure of transfer of passive immunity. In certain aspects, colostrum management information may include information regarding one or more risk factors listed in Table 9, Section III. In certain aspects, the two or more, three or more, four or more, five or more, six or more, seven or more, eight or more, nine or more, ten or more, or all eleven items of colostrum management information may be extracted.

In certain aspects, the dairy farm management practice information comprises one or more items of milk feeding information. In certain aspects, the management practice category of milk feeding includes activities that constitute the pre-weaned calf's milk nutrition. In certain aspects, the one or more items of milk feeding information comprises one or more of (e.g., 1, 2, 3, 4, 5, 6, or each of): feeding order by age and health of calves, the source of milk, whether the milk is pasteurized, whether the milk is tested for bacteria before or after pasteurization, whether the milk is supplemented with replacer, how much milk is fed each day, and how often milk is fed each day. In certain aspects, milk feeding management information may include information regarding one or more risk factors listed in Table 9, Section IV. In certain aspects, two or more, three or more, four or more, five or more, six or more, or all seven items of milk feeding management information may be extracted.

In certain aspects, the dairy farm management practice information comprises one or more items of vaccination information. In certain aspects, the management practice category of vaccinations may include immunization protocols for respiratory pathogens in pre-weaned calves and their dams. In certain aspects, the one or more items of vaccination information comprises one or more of (e.g., 1, 2, or each of): whether vaccines for one or more respiratory pathogens were given, modified live or killed to calves, and modified live or killed to cows. In certain aspects, vaccinations management practice information may include information regarding one or more risk factors listed in Table 9, Section V. In certain aspects, one or more, two or more, or all three items of vaccination information may be extracted.

In certain aspects, the dairy farm management practice information includes one or more items of calf housing information. In certain aspects, the management practice category of calf housing includes factors that determine a pre-weaned calf's housing environment. In certain aspects, the one or more items of calf housing information includes one or more of (e.g., 1, 2, 3, 4, 5, 6, 7, 8, 9, or each of): the type of driving surfaces next to calf hutches, the presence of dust abatement measures, whether dust was perceived as a problem, whether any pre-weaned calves were housed in groups, the presence of an extra shade structure over the hutches, the presence of calf-to-calf contact in calves>75 days old, hutch wall material, hutch floor material, the presence and type of manure flush system under hutches. In certain aspects, calf housing management practice information may include information regarding one or more risk factors listed in Table 9, Section VI. In certain aspects, one or more, two or more, three or more, four or more, five or more, six or more, seven or more, eight or more, nine or more, or all ten items of calf housing management practice information may be extracted.

In certain aspects, the dairy farm management practice information includes one or more items of information for three or more, four or more, five or more, or each of the dairy farm management practice categories selected from the group consisting of: basic herd risk profile, maternity pen management, colostrum management, milk feeding, vaccinations, and calf housing.

In certain aspects, administering a questionnaire comprising a plurality of questions regarding management practices in a dairy farm may include self-administering the questionnaire, for example, entering information regarding management practices in a dairy farm into a computer (e.g., a tablet computer, a mobile phone, a laptop computer, or a desktop computer). In certain aspects, extracting the practice information may include providing the recorded information provided in response to the questionnaire in a form that is suitable for calculations while retaining the link between the question and the corresponding response as well as which item of management practice information the question pertains to.

In certain aspects, the one or more items of information for two or more dairy farm management practice categories extracted may be assigned weighted risk scores. In certain aspects, the assigning weighted risk scores to the extracted dairy farm management practice categories items may include multiplying the items of extracted information with a factor that reflects the magnitude of the association of the item of information to risk of occurrence of BRD. In certain aspects, the factor may be determined from analysis of data correlating farm management practices to BRD prevalence and/or incidence in pre-weaned calves. In certain aspects, the higher the correlation of an item of information to risk of occurrence of BRD, the higher the factor. In certain aspects, certain items of information may be assigned as a reference to which the remainder of items of information are compared. The reference item may be assigned a weighted score of 0. For example, in the dairy farm management practice category of basic herd risk profile, the item of information of a herd size of 1000-3999 milking cows may be assigned a weighted score of 0. Other examples of items of information that can be referenced for the remainder of items of information in a particular management practice information category are shown in Tables 2-7. In certain aspects, the factor for assigning weighted risk scores to the extracted dairy farm management practice categories items may be calculated using hazard ratios (HR). In certain aspects, the factor for assigning weighted risk scores to the extracted dairy farm management practice categories items may be calculated using odds ratios (OR). These ratios reflect the magnitude of the association of variables with the binary outcome presence of BRD in a calf and may be calculated by performing univariable or multivariable analysis of variables based on responses to questions regarding items of information in a particular management practice category. In certain aspects, data on dairy management practices may be collected via a questionnaire covering the areas of maternity pen management (pen density, individual versus group pen calving, bedding type and hygiene practices), colostrum management (colostrum source, heat-treatment, storage time, temperature and container type, quality control, volume fed, testing of calves for failure of transfer of passive immunity), nutrition, (feeding order, milk source, pasteurization, quality control, volume fed), vaccinations (type of vaccines administered to calves and cows), and calf housing (dust abatement, housing materials, calf-to-calf contact, presence of extra shade, manure flush systems).

In certain aspects, the factor by which an extracted item of information for a dairy farm management practice category may be multiplied for assigning weighted risk score to the item may be a regression coefficient calculated for that item using HR or OR. In certain aspects, the assigning weighted risk scores to the extracted dairy farm management practice categories items may include multiplying the items of extracted information with the corresponding regression coefficient listed in Tables 2-7.

In certain aspects, the method may further include determining a total risk score for each of the two or more dairy farm management practice categories based on the assigned weighted risk scores. In certain aspects, determining a total risk score for each of the two or more dairy farm management practice categories may include adding the assigned weighted risk scores in each dairy farm management practice category. In certain aspects, at least two items of each management practice information may be assigned weighted risk scores and the weighted risk scores are added to obtain a total risk score of each management practice information.

In certain aspects, the method may further include the step of determining the risk of BRD in pre-weaned dairy calves on the dairy farm based on the total risk scores for the two or more dairy farm management practice categories. In certain aspects, the method may include adding the total risk scores for the two or more dairy farm management practice categories to determine a grand total of the total risk scores and using the grand total for determining the risk of BRD in pre-weaned dairy calves on the dairy farm.

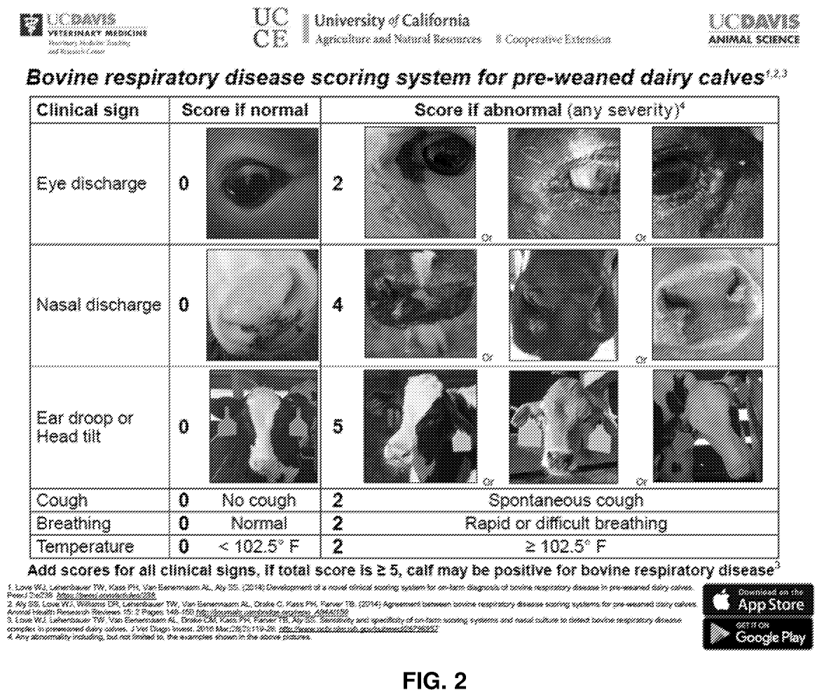

In certain aspects, a higher risk score in a dairy farm management practice category may be indicative of increased risk of BRD. In certain aspects, the method may further include recording the total risk scores and a prevalence score taken on a random sample of calves for BRD for the dairy farm and using the correlation as a reference point for determining the effect of changes in one or more management practice changes. In certain aspects, the prevalence score for BRD may be determined by using a CA BRD scoring system (FIG. 2) or any other methodology that produces an unbiased measure of the disease (BRD) burden in the cohort of calves assessed for risk of BRD.

In certain aspects, the method may further include recommending to the dairy farm one or more management practice changes based on the extracted dairy farm management practice information. In certain aspects, recommending to the dairy farm a management practice change may be based upon a higher total risk score for that dairy farm management practice category. In certain aspects, the method may further comprise calculating a new total risk score for that dairy farm management practice category after changes are implemented, and assessing the effect of the changes. In certain aspects, the method may further comprise determining a second prevalence estimate along with the new total risk score and verifying whether changes to management practices have resulted in the desired change in BRD prevalence.

In certain aspects, the dairy farm may be in a geographic location having climate similar to the climate in California. In certain aspects, the dairy farm may be located in a place having a climate similar to California climate Zone2, 12 or 13. In certain aspects, the dairy farm may be located in California. In certain aspects, the dairy farm may be located in a location in California having a California climate Zones 2, 12 or 13. In certain aspects, the dairy farm may be located in Northern California, Northern San Joaquin Valley, or Greater Southern California.

In certain aspects, the method may further include producing a report comprising the total risk scores for the two or more dairy farm management practice categories. In certain aspects, the report presents the total risk scores on a scale in context with the total risk scores of other dairy farms. In certain aspects, the report further comprises the risk of BRD in pre-weaned dairy calves on the dairy farm based on the total risk scores for the two or more dairy farm management practice categories. In certain aspects, the report presents the risk of BRD on a scale in context with the risk of BRD of other dairy farms. In certain aspects, the other dairy farms may be farms in the same geographic location (e.g., in California) and/or in the same climate.

In certain aspects, producing a report includes displaying the total risk scores with or without a scale in context with the total risk scores of other dairy farms and/or the risk of BRD with or without a scale in context with the risk of BRD of other dairy farms on a display (e.g., a display of a desktop computer, laptop computer, television, tablet computer, smartphone, or the like) or printout. In some embodiments, the display may be of the same computer device used to implement the method for determining the risk of BRD. Alternatively or additionally, the report may be displayed on the display of a computer device other than the computer device used to produce the implement the method.

In certain aspects, the method may be a computer implemented method. In certain aspects, the method may be implemented using a computer system, such as, a tablet computer, a mobile phone, a laptop computer, or a desktop computer.

### Non-Transitory Computer-Readable Media and Computer Devices

As summarized above, the present disclosure also provides a non-transitory computer-readable medium for practicing the methods provided herein. In certain aspects, the non-transitory computer-readable medium may include instructions which, when executed by a computer system, cause the computer system to extract dairy farm management practice information from a dairy farm, wherein the dairy farm management practice information comprises one or more items of information for two or more dairy farm management practice categories selected from the group consisting of: basic herd risk profile, maternity pen management, colostrum management, milk feeding, vaccinations, and calf housing; assign weighted risk scores to the items of dairy farm management practice information extracted; and determine a total risk score for each of the two or more dairy farm management practice categories based on the assigned weighted risk scores.

In certain aspects, the instructions, when executed by a computer system, cause the computer system to extract the dairy farm management practice information by prompting an individual to input the dairy farm management practice information into the computer system via an input device.

The dairy farm management practice information may be as described in the Methods section hereinabove, which is incorporated but not reiterated herein for purposes of brevity. For example, the dairy farm management practice information may be as described in the preceding Methods section and may include one or more items of information for two or more dairy farm management practice categories selected from the group consisting of: basic herd risk profile, maternity pen management, colostrum management, milk feeding, vaccinations, and calf housing, as described in the preceding section.

The input device may be any suitable input device. In certain aspects, the input device is selected from the group consisting of: a touchscreen display, a mouse, a keyboard, and any combination thereof.

In certain aspects, the instructions, when executed by a computer system, further cause the computer system to determine the risk of BRD in pre-weaned dairy calves on the dairy farm based on the total risk scores for the two or more dairy farm management practice categories.

In certain aspects, the instructions, when executed by a computer system, further cause the computer system to recommend to the dairy farm one or more management practice changes based on the extracted dairy farm management practice information.

In certain aspects, the instructions, when executed by a computer system, further cause the computer system to produce a report comprising the total risk scores for the two or more dairy farm management practice categories. In certain aspects, the report presents the total risk scores on a scale in context with the total risk scores of other dairy farms.

In certain aspects, the report further comprises the risk of BRD in dairy calves on the dairy farm based on the total risk scores for the two or more dairy farm management practice categories.

In certain aspects, the report presents the risk of BRD on a scale in context with the risk of BRD of other dairy farms. In certain aspects, the non-transitory computer-readable medium is present on a local computer device. In certain aspects, the non-transitory computer-readable medium is present on a remote server.

Instructions can be coded onto a non-transitory computer-readable medium in the form of “programming”, where the term “computer-readable medium” as used herein refers to any non-transitory storage or transmission medium that participates in providing instructions and/or data to a computer for execution and/or processing. Examples of storage media include a hard disk, optical disk, magneto-optical disk, CD-ROM, CD-R, magnetic tape, non-volatile memory card, ROM, DVD-ROM, Blue-ray disk, solid state disk, network attached storage (NAS), etc., whether or not such devices are internal or external to the computer. A file containing information can be “stored” on computer readable medium, where “storing” means recording information such that it is accessible and retrievable at a later date by a computer.

The instructions may be in the form of programming that is written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as many others.

The present disclosure also provides computer devices. The computer devices include one or more processors and any of the non-transitory computer readable media of the present disclosure. Accordingly, in some embodiments, the computer devices are capable of performing any of the methods described in the Methods section herein.

In certain aspects, a computer device of the present disclosure is a local computer device. In some embodiments, the computer device is a remote computer device (e.g., a remote server), meaning that the instructions are executed on a computer device different from a local computer device and/or the instructions are downloadable from the remote computer device to a local computer device, e.g., for execution on the local computer device. In some embodiments, the instructions constitute a web-based application stored on a remote server. The following examples are offered by way of illustration and not by way of limitation.

### Experimental

Introduction. A novel approach in reducing the burden from BRD in pre-weaned calves may be a comprehensive on-farm risk assessment to identify and mitigate dairy specific factors that may lead to BRD in calves, to find ways to improve calf immunity, and to strengthen calf health. The multifactorial nature of the disease complex, where stress allows opportunistic commensal bacteria to overwhelm the hosts' immune system, lends itself to a risk assessment approach (Gorden and Plummer, 2010). In addition, a risk assessment approach to prevent BRD in dairy calves may reduce the use of antimicrobials and support the goals outlined in the National Action Plan for Combating Antibiotic-Resistant Bacteria, in particular, goal 1.3 “Identify and implement measures to foster stewardship of antibiotics in animals”(National Action Plan for Combating Antibiotic-Resistant Bacteria, 2015).

The objective of this study was to design a new tool for assessing the present herd risk, as well as identifying areas of possible risk mitigation, in a field-ready format. The risk assessment questionnaire presented here is intended to be used in combination with the California BRD scoring system for pre-weaned dairy calves (Love et al., 2014) that will allow the user to follow herd prevalence before and after interventions. With the daily influx of newborn calves, changes in prevalence may be observed within months of implementation of the prevention or control program, further justifying the use of a risk assessment approach with BRD prevention and control.

Abstract. Due to the stagnant morbidity and mortality of bovine respiratory disease (BRD) in dairy calves as well as an increasing urgency for the judicious use of antimicrobials in farm animals, a comprehensive risk assessment tool for BRD in pre-weaned dairy calves has been designed based on several longitudinal and cross-sectional studies. As a multifactorial disease complex where stressors on the immune function of the host play a large role in susceptibility to this disease, BRD lends itself to a risk management approach. Areas of known or suspected impact on BRD in pre-weaned calves had been explored in two large studies correlating management factors to BRD prevalence (BRD 100 study) or incidence (BRD 10K study), which built the backbone of the scores presented here. Preference was given to results from multivariable over univariable analyses. When used, univariable estimates were adjusted for confounders or stratified by effect modifiers if necessary. Regression coefficients were translated into scores, which are presented in a field-ready tool consisting of a questionnaire and prevalence estimate for BRD in the calf herd. Scores for 100 dairies across California were used to benchmark a dairy's risk on a spectrum. The resulting risk assessment tool allows the user to objectively monitor BRD prevalence and adjust management factors that may be the most important contributors to the risk on farm.

### Materials and Methods

### Study Design

The results of two studies on the association between management factors on dairies and BRD build the foundation for the risk scores used in the tool: “Management factors associated with Bovine Respiratory Disease (BRD) in Pre-weaned Calves on California Dairies: the BRD 100 study” (Maier et al., 2019) and “Epidemiology of Bovine Respiratory Disease in Pre-weaned Calves on California Dairies, the BRD 10K study” (Dubrovsky et al., 2019). In both the BRD 100 and the BRD 10K studies, data on dairy management practices was collected via a questionnaire covering the areas of maternity pen management (pen density, individual versus group pen calving, bedding type and hygiene practices), colostrum management (colostrum source, heat-treatment, storage time, temperature and container type, quality control, volume fed, testing of calves for failure of transfer of passive immunity), nutrition, (feeding order, milk source, pasteurization, quality control, volume fed), vaccinations (type of vaccines administered to calves and cows), and calf housing (dust abatement, housing materials, calf-to-calf contact, presence of extra shade, manure flush systems) (Table 1).

TABLE 1
Categories and subcategories of information collected during a
cross-sectional study (BRD 100) correlating farm management
practices on 100 California dairies to BRD prevalence in preweaned
calves or a longitudinal study (BRD 10K) correlating farm management
practices on five California dairies to BRD incidence in preweaned calves.

| Maternity | | | | |
|---|---|---|---|---|
| pen | Colostrum | | | |
| management | management | Nutrition | Vaccinations | Housing |
| Pen density | Source | Feeding order | Type of | Dust |
| Individual | Heat treatment | Milk source | vaccines | abatement |
| versus group | Storage time | Pasteurization | administered | Housing |
| pen calving | Storage | Quality | to calves | materials |
| Bedding | temperature | control | and cows | Calf-to-calf |
| type | Container type | Volume fed | | contact |
| Hygiene | Quality control | | | Presence of |
| practices | Volume fed | | | extra shade |
| | Testing calves | | | Manure |
| | for failure of | | | flush |
| | transfer of | | | systems |
| | passive | | | |
| | immunity | | | |

The BRD 100 study correlated management factors to point prevalence estimates of BRD in a random sample of pre-weaned calves taken during visits to a convenience sample of 100 dairies across California, which took place between May 2014 and April 2016. The cross-sectional study approach allowed for enrolling a large number of dairies across the state which captured the variability in practices across California's dairy industry and hence provides for greater external validity.

In the BRD 10K study, which enrolled a convenience sample of five dairies between March and August, 2015, and collected data on pre-weaned calves for at least one year on each dairy, over 11,000 calves were followed from birth to weaning. Trained dairy personnel recorded BRD diagnoses and treatments for pre-weaned calves and incidence of BRD was correlated to management practices on the dairies. The prospective cohort study approach allowed for estimating risk of BRD conditional on the different management practices.

### Risk Assessment Score Estimation

Scores in the risk assessment tool are based on regression coefficients (Moons et al., 2002) from univariable or multivariable analysis of variables based on responses to the questionnaire used in both the BRD 100 and BRD 10K studies. The questionnaire was based on multivariate analysis of a state-wide mail survey of management practices that are known to be correlated with calf health and specifically BRD in pre-weaned dairy calves (Love et al., 2016a). In the BRD 10K study, shared frailty models, which are extensions of the Cox proportional hazards (CPH) model, assessed the hazard of exposure to management factors on the incidence of BRD in study calves. The effect measure in the BRD 10K was the hazard ratio (HR). In the BRD 100 study, generalized linear mixed models with a logit link function and dairy as the random effect were used to estimate the magnitude of the association of explanatory variables with the binary outcome presence of BRD in a calf. The effect measure calculated in the BRD 100 study was the odds ratio (OR).

The order of preference was to use multivariable results from the longitudinal study (BRD 10K), followed by multivariable results from the cross-sectional study (BRD 100), then univariable longitudinal, and finally univariable cross-sectional results. Results for the longitudinal study were not always available, because of lack of variability between enrolled dairies. If a regression coefficient for a variable was statistically non-significant (P 0.05) in one model but statistically significant in a different model (e.g. longitudinal univariable versus cross-sectional univariable) preference was given to the significant result. If univariable estimates were used they were assessed for known confounders or split into strata specific estimates in the case of significant effect modification. All reference levels were chosen so that only positive scores would be obtained for ease of score addition. Regression coefficients were multiplied by 100 to obtain whole number scores. In a few instances, the average of regression coefficients from logistic regression and CPH models was used, when both P-values were <0.05. Higher scores represent higher risk.

The risk assessment tool was divided into scores comprising the basic herd risk profile, maternity pen management, colostrum management, milk feeding factors, vaccinations and housing factors. The basic herd risk profile consists of those factors that are inherent in the herd or farm and that are unlikely to change in the intermediate or even long term. Components of the basic herd risk profile are herd size, breed distribution, and organic herd status. Region of the farm may also be included in determining basic herd risk profile. Season was also part of the basic herd risk profile, as season was significantly associated with BRD in both studies. Obviously, seasons change over time; however, inclusion of season was deemed appropriate to adjust the baseline risk for the season during which a risk assessment is conducted.

Maternity management factors included the frequency of bedding changes in the maternity pen combined with individual or group pen calving for cows and heifers, whether cows and heifers calve together, the materials used for bedding, whether calves are born on pasture, and whether at least 95% of calves are removed from the dam within an hour of birth.

Factors related to colostrum management included the source of colostrum, whether colostrum was supplemented with replacer, whether it was heat-treated before feeding, and how long it was stored before heat-treatment. In addition, testing for bacterial content of colostrum, use of colostrum from first calf heifers, addition of a preservative, the type of storage container and testing for immunoglobulin content were included. Finally, whether calves received at least 2.84 L (3 quarts) of colostrum in the first 12 hours of life and whether calves were tested for failure of transfer of passive immunity was included under colostrum management.

Milk feeding factors included the feeding order by age and health, the source of milk, whether the dairy pasteurizes milk fed to calves, whether the milk is tested for bacteria before or after pasteurization, whether it is supplemented with replacer, how much milk and how often milk is fed each day.

Vaccinations were assessed by whether each of the following vaccines for respiratory pathogens were given: intranasal or any injectable (modified live or killed) to calves and modified live or killed to cows.

Lastly, housing factors included the type of driving surfaces next to calf hutches, whether any dust abatement measures were in place, whether dust was perceived as a problem, whether any pre-weaned calves were housed in groups, whether there was an extra shade structure over the hutches, whether calf-to-calf contact in calves >75 days old was possible, material hutch walls or floors were made out of, and whether and what type of manure flush system under hutches was in place.

### Benchmarking

Each of the above sections was assigned a maximum possible score. The dairies participating in the BRD 100 study were assigned scores based on their answers to the questionnaire as part of that study. The range of scores from 100 dairies in California was divided into the following categories as benchmarks: very low for the 10th percentile, low for the 25^{th} percentile, moderate for the 50^{th} percentile, high for the 75^{th} percentile and very high for the 90^{th} percentile of scores. The range of scores for the 100 dairies, which puts the score obtained for a single dairy under investigation into perspective, was included after each section and for the total score. For some questions, contingencies were added to point assignments in the risk assessment tool, such as question 4.5 “Is milk tested for bacteria before pasteurization or before feeding?”, with possible answers: “No; Yes and we never find bacteria; Yes and if bacteria are found, protocols are reviewed or other action taken; Yes and no particular action taken if bacteria are found”. In the survey of 100 dairies, however, only a yes/no answer was recorded for the question. Where this was the case, questions were not counted toward the benchmark scores. Other questions not included in benchmarking for the same reason are 3.4 “Do you use colostrum from first calf heifers?”, 3.5 “Do you add a preservative before storing colostrum?”, 3.7 “Is colostrum tested for IgG?”, 3.9 “Are calves tested for transfer of passive immunity?”, 4.6 “Is milk ever tested for bacteria after pasteurization?” and 6.2 “Is there any dust abatement next to the calf hutches?”.

A section to record the calf herd's BRD point prevalence at time of completing the risk assessment based on the CA BRD scoring system is included. Finally, a form to record what is feasible from the recommended management changes based on a discussion with the herd owner or manager follows the risk assessment questionnaire (Love et al., 2014, Love et al., 2016b).

Results and Discussion. The risk assessment tool presented here is a novel comprehensive approach for control and prevention of BRD in preweaned calves on dairies. Risk scores are based on regression coefficients from analysis of two data sets that were collected as part of a cross-sectional and a longitudinal study on the association of dairy management factors with BRD in preweaned dairy calves. The availability of weighted scores will allow the investigator, or possibly the producer or herd veterinarian, to identify the most effective management changes that may affect BRD prevalence in the calf herd on their specific dairy. The goal of the risk assessment tool is to calculate a score based on the herd risk profile and the management practices in place on the farm assessed by adding all the individual scores. The section scores, and the final score create benchmarks for each management component of the risk assessment tool and the overall BRD risk in preweaned calves on the farm assessed and should be documented in combination with a prevalence estimate. The risk assessment section for recommended changes provides space to assign duties to dairy staff as well as a comments section to track changes. After changes are implemented, the effect of the changes can be assessed once a new cohort of calves is on the farm and has been exposed to the new management practices. Hence, follow-up risk assessments are highly recommended to verify that the management changes agreed upon were completed and to revisit or resolve hurdles for those changes that were not accomplished. Similarly, preweaning calves' BRD prevalence in the assessed herd should be estimated at the time of each risk assessment to verify that the BRD control and prevention efforts were effective.

A true random sample of calves from the herd of the appropriate size should be pursued for each prevalence estimation to allow for a fair comparison of BRD prevalence over time. To facilitate prevalence estimation, the use of the CA BRD Scoring tool is recommended, which is freely available in English, Spanish and Arabic as iOS (Apple Inc. Cupertino, Calif.) and Android (Mountain View, Calif.) applications for smart phones and tablets. The application guides the user through which calves to score, adds up scores in individual calves to determine positive or negative status and calculates prevalence including a 95% confidence interval.

Basic Herd Risk Profile

Table 2 shows the regression coefficients corresponding to the basic herd risk profile section and their sources in terms of study design and statistical models. All regression coefficients are from the BRD 100 study, as the BRD 10K study only contained five dairies and did not offer enough variability to explore these variables. The 100 study dairies that contributed to this section were spatially distributed in all the dairy counties which helped capture the variation in the state's climate. California's climate is primarily subtropical Mediterranean and includes several microclimates that vary widely between the coast and inland regions, mountainous terrain and lowland valleys. Similarly, given that the studies were conducted over two years with continuous enrollment year-round, the seasonal differences in the state's climate with mild winters, varying annual rainfall and warm to hot summers were similarly well-represented. Hence, the spatial and temporal variability in climate over the study period captures a wide variety of climates representing many locations where dairies operate.

Although herd size was included in the multivariable BRD 100 model to adjust for confounding, it was not a significant predictor in that model and thus estimates from univariable analysis were included for risk assessment (OR 2.78, P=0.044 for herd size<250 versus 1,000-3,999;). California dairies are on average larger than the average US dairy. According to the California Department of Food and Agriculture the average dairy herd size in 2017 in California was 1,304 milking cows (California Department of Food and Agriculture, 2017) while the latest report on dairy cattle management practices published by the USDA categorizes herd sizes with more than 500 cows as large (USDA, 2016) The lower scores calculated for herd sizes between 1,000 and 3,999 milking cows may be because such dairies typically have more specialized labor for the care of calves. The effect of specialized labor for care of calves may attenuate with increasing numbers of calves, but no studies are available to explore this effect. One study found a higher risk of preweaned calf pneumonia in Ottawa for increasing number of calvings per farm per year, however farm sizes were small and ranged between 23 and 154 calvings per year (Waltner-Toews, 1986).

Organic herd status was not a significant predictor in any of the models and an estimate from a univariable analysis was included (OR 1.58, P=0.25 for organic versus conventional herds). Breed was included to adjust for its biological effect in the BRD 100 multivariable model and scores are based on those coefficients (OR 2.04, P=0.37 for Holstein versus mixed or other breed, OR 2.53, P=0.30 for Jersey versus mixed or another breed). The minimum score possible for the basic herd risk profile is 0 for a medium to large sized dairy (1,000-3,999 milking cows) with breeds other than pure Jersey or pure Holstein with conventional management in the spring while the maximum score possible is 103 for an organic small Jersey herd (<250) in in the fall.

TABLE 2
Regression coefficients for basic herd risk profile factors for use in a
risk assessment tool for bovine respiratory disease (BRD) in preweaned
dairy calves. Regression coefficients are from analysis of data in a
cross-sectional study correlating farm management practices on
100 California dairies to BRD prevalence in preweaned calves.

| | | | | | |
|---|---|---|---|---|---|
| | Regression | Standard | Odds | | |
| Factor | coefficient | error | ratio | p-value | Source* |

Herd size (milking cows)

| | | | | | |
|---|---|---|---|---|---|
| <250 | 1.02 | 0.51 | 2.78 | 0.04 | BRD 100, UV |
| 250-999 | 0.57 | 0.36 | 1.77 | 0.11 | |
| 1,000-3,999 | Ref. | | | | |
| 4,000+ | 0.76 | 0.60 | 2.14 | 0.21 | |

Organic status

| | | | | | |
|---|---|---|---|---|---|
| no | Ref. | | | | |
| yes | 0.46 | 0.39 | 1.58 | 0.25 | BRD 100, UV |

Season

| | | | | |
|---|---|---|---|---|
| Spring | Ref. | | | |
| (Apr-Jun) | | | | |
| Summer | 1.27 | 3.57 | 0.10 | BRD 100, MV |
| (Jul-Sep) | | | | |
| Fall | 1.95 | 7.04 | 0.047 | |
| (Oct-Dec) | | | | |
| Winter | 1.12 | 3.05 | 0.22 | |
| (Jan-Mar) | | | | |

Breed

| | | | | | |
|---|---|---|---|---|---|
| Crossbred | Ref. | | | | |
| or other | | | | | |
| purebred | | | | | |
| Holstein | 0.71 | 0.80 | 2.04 | 0.37 | BRD 100, MV |
| Jersey | 0.93 | 0.89 | 2.53 | 0.30 | |

*BRD 100: Study on prevalence of bovine respiratory disease in 4,636 preweaned calves or calves housed in hutches on 100 dairies in California surveyed between May 2014 and April 2016
UV = univariable analysis
MV = multivariable analysis

Maternity Pen Management

Variables and their regression coefficients corresponding to maternity pen management are similarly shown in Table 3 along with their study design and statistical model sources. In univariable models, the variable for whether cows or heifers calve individually, versus those calving in groups or a mix of individual and group calving, was not significantly associated with BRD in calves. The effect of calving in groups or individually on calf health could be mitigated by the frequency of bedding changes which is why an interaction term between the number of bedding changes per month and group or individual calving was explored. With an indicator variable for high (>5 times) versus low 5 times) maternity pen bedding change frequency per month as an effect modifier, cows calving in group pens where bedding was changed only up to 5 times per month was positively associated with BRD (OR 3.53, P=0.046) in the calves compared to calving pens with higher bedding change frequencies. A score of 0 was assigned to calving pens for dams when bedding was changed more than 5 times per month. Cows and heifers calving in the same pen was assessed in univariable analysis using data from the BRD 100 study, resulting in an OR of 1.37 (P=0.39) for BRD in calves compared to when cows and heifers calve separately.

In a BRD 100 univariable model (cross-sectional study) where bedding material was coded with manure as the reference category and sand or dirt, plant material (straw, almond shells, etc.) and gypsum as indicator variables, the odds of BRD were lower for calves where gypsum (OR 0.66, P=0.79) was used as bedding material in the maternity pen compared to dry manure. The decision to combine dried manure and gypsum into one category with the lowest risk was made because the use of gypsum as bedding material in our study population was rare. Gypsum, used by 2 study dairies as bedding after being acquired as recycled drywall, could have hydroscopic properties that may minimize the microbial load in the maternity pen bedding. Further research is needed to document the superior microbiological properties of this type of bedding compared to other materials. Comparing dirt (OR 3.94, P=0.01) or plant material (OR 1.65, P=0.37) as bedding materials to dried manure or gypsum resulted in higher odds of BRD in calves compared to the reference category. In contrast, use of sand as a bedding material produced a protective univariable odds ratio of 0.40 (P=0.63). An in vitro study comparing organic and inorganic bedding materials showed no difference in growth rates of Enterococcus faecum in recycled sand versus digested manure solids within the first 72 hours of inoculation (Godden et al., 2008). A univariable model from the BRD 100 study comparing odds of BRD depending on whether or not cows calve on pasture was stratified by whether or not ≥95% of calves are removed from the dam within an hour of birth. Those calves where fewer than 95% were removed from dams within an hour had an OR of 2.63 (P=0.001) if they were born on pasture compared to those not born on pasture. The effect of pasture calving on the health of calves may be mostly related to the time it takes to remove the calves from the dam. Stratification of pasture calving by whether or not 95% of calves are removed from their dams within one hour showed that none of the 72 calves born on pasture sampled on dairies where ≥95% of calves are removed from their dams within one hour were BRD score positive. However, these calves represent only 1 dairy, so the association may be confounded by other factors. The importance of the prompt removal of calves from dams to prevent exposure of newborns to pathogens may be further emphasized by the fact that calves on dairies where most calves are removed from dams within an hour have lower odds of BRD than those calves on dairies that leave them with the dam longer. Results for whether ≥95% of calves were removed within one hour from dams regardless of maternity pen environment also stem from the BRD 100 data in univariable analysis, with an OR of 3.25 (P=0.01) for BRD for those calves on dairies that remove fewer calves compared to those removing ≥95% of calves within one hour of birth.

TABLE 3
Regression coefficients for maternity pen management factors for use in a risk
assessment tool for bovine respiratory disease (BRD) in preweaned dairy calves. Regression
coefficients are from analysis of data in a cross-sectional study correlating farm management
practices on 100 California dairies to BRD prevalence in preweaned calves.

| | Regression | Standard | Odds | | |
|---|---|---|---|---|---|
| Factor | coefficient | error | ratio | p-value | Source* |
| Cows and heifers calve | | | | | |
| together | | | | | |
| No | Ref. | | | | BRD 100, UV |
| Yes | 0.32 | 0.37 | 1.37 | 0.39 | |
| > 5 bedding changes per | Ref. | | | | |
| month | | | | | |
| If 5 or fewer bedding changes | | | | | |
| per month and | | | | | BRD 100, UV |
| ≥ 95% cows calve | 1.56 | 1.13 | 4.75 | 0.17 | |
| individually | | | | | |
| ≥ 95% cows calve in | 1.26 | 0.63 | 3.53 | 0.046 | |
| groups | | | | | |
| Mix of individual and | 0.32 | 0.77 | 1.37 | 0.68 | |
| group calving for cows | | | | | |
| Bedding materials in calving | | | | | |
| pens | | | | | |
| Dried manure, gypsum | Ref. | | | | |
| Sand | −0.90 | 1.85 | 0.40 | 0.63 | BRD 100, UV |
| Dirt | 1.37 | 0.55 | 3.94 | 0.01 | |
| Plant material² | 0.50 | 0.56 | 1.65 | 0.37 | |
| Pasture calving and <95% of | | | | | |
| calves removed from dam | | | | | |
| within 1 hour of calving | | | | | |
| No | Ref. | | | | |
| Yes | 0.97 | 0.30 | 2.63 | 0.001 | BRD 100, UV |
| Are ≥95% of calves removed | | | | | |
| from dam within 1 hour of | | | | | |
| calving | | | | | |
| Yes | Ref. | | | | |
| No | 1.18 | 0.47 | 3.25 | 0.01 | BRD 100, UV |

*BRD 100: Study on prevalence of bovine respiratory disease in 4,636 pre-weaned calves or calves housed in hutches on 100 dairies in California surveyed between May 2014 and April 2016
UV = univariable analysis
²Rice hulls, almond shells, straw, wood shavings, wood chips

### Colostrum Management

Table 4 summarizes the colostrum management variables and their sources. Unless noted in Table 4, all analyses are from univariable analyses in the BRD 100 study. The variable for source of colostrum assigns risk points to calves receiving pooled colostrum (OR 1.72, P=0.31) and those receiving colostrum from a mix of sources including colostrum replacer (OR 2.28, P=0.12) versus those receiving colostrum from an individual cow, which is the reference category. Feeding pooled colostrum has been thought to lead to dilution of immunoglobulin concentration and increased chances of disease transmission to calves because multiple cows are represented in a single colostrum feeding (McGuirk and Collins, 2004). An interaction term between nursing from the dam and pasture calving, in order to incorporate the potential added risks of pasture calving for calf health, resulted in an OR of 1.29 (P=0.67) for BRD in calves born on pasture versus those that were not born on pasture in calves that nursed from the dam. The points assigned for supplementing colostrum with colostrum replacer was based on the HR of 1.55 (P=0.006) for BRD in calves on dairies with that practice obtained from univariable analysis in the BRD 10K study. However, a score indicating higher risk for supplementing colostrum should only be assigned if the dairy does not routinely check for IgG levels in colostrum and supplements colostrum routinely. The act of supplementing colostrum without testing may be associated with mitigating poor colostrum management and BRD in calves, which is why the score should only apply to those dairies that don't test for IgG levels. The score for not heat-treating colostrum is based on an OR of 2.37 (P=0.04) for BRD in calves on dairies not heat-treating versus those that do. The benefits of heat-treatment of colostrum have recently been confirmed in a large study of factors associated with colostrum quality and passive immunity transfer status of dairy heifer calves (Shivley et al., 2018). The score for how long colostrum is stored before heat-treatment was based on an OR for BRD in calves of 2.84 (P=0.15) for calves from dairies where colostrum is stored for longer than 10 hours before heat-treatment versus those where it is stored only up to 10 hours. A bimodal distribution of storage times for colostrum before heat treatment in the BRD 100 study led to dichotomization of this variable into 10 hours and >10 hours. Increased storage times for colostrum may lead to higher bacterial loads, depending on initial loads and storage temperature, which is also related to the next variable, whether testing of colostrum for bacterial content before heat treating is performed (McGuirk and Collins, 2004). The OR for BRD in calves on dairies where colostrum is not tested for bacteria before heat-treatment versus those where colostrum is tested for bacteria before heat-treatment was 1.60 (P=0.25). For dairies using colostrum from first lactation cows the OR for BRD in calves was 1.18 (P=0.64). Serum IgG levels of calves receiving colostrum from first lactation cows in a recent study were actually highest compared to serum IgG levels in calves fed higher lactation cow colostrum (Shivley et al., 2018). However, colostrum samples from third or greater lactation dams had the highest quality in the Shivley study. The contribution of first lactation cow colostrum to the risk score is small and discarding first lactation cows' colostrum or substituting it with colostrum replacer if there is a shortage of colostrum is not recommended. While testing of all colostrum for IgG content is recommended, first lactation cows' colostrum should be tested in any case given the higher odds of BRD in calves fed the latter compared to feeding only higher lactation cow colostrum.

The HR for BRD in calves on dairies where a preservative such as potassium sorbate is added to colostrum was 1.29 (P=0.77). Adding preservative to colostrum per se is likely not a cause of BRD in calves, which is why the score for this factor should only be assigned for dairies adding preservative because of past problems with colostrum quality. It is important to understand that the root cause of increased BRD risk associated with preservative use in colostrum in our study likely stems from hygiene problems during colostrum harvest and storage, which should be discussed during a risk assessment. A study comparing bacterial counts of colostrum samples stored at room temperature and refrigerated with or without potassium sorbate addition found lowest total plate count and total coliform counts in refrigerated samples treated with potassium sorbate (Stewart et al., 2005). The use of a solid storage container, such as a plastic bottle, versus a bag resulted in an OR of 1.82 (P=0.20) for BRD in calves. Storing frozen colostrum in bags may result in a more even freezing and thawing process and thus less damage to proteins. Not testing colostrum for IgG levels was associated with BRD in the BRD 10K study with a HR of 3.10 (P=0.11). However, it is not the testing for IgG levels in colostrum itself that prevents BRD, but the actions that should follow a low-test result. The risk assessment tool therefore assigns points for those dairies that do not test colostrum for IgG levels as well as those dairies that do not have a follow-up protocol for low IgG level results, such as colostrum supplementation of low quality colostrum. Calves receiving less than 2.84 L (3 quarts) of colostrum versus those that received at least 2.84 L in the first 12 hours of life had an OR of 1.32 (P=0.58) for BRD. The cut-point of 3 quarts was chosen based on a study where 3 L of colostrum fed to calves provided adequate serum total protein levels at 24 hours of age, while 1.5 L of colostrum provided marginal serum total protein levels (Godden et al., 2009). A different cut-point of 4 quarts of colostrum fed in the first 12 hours as well as stratification by breed and controlling for breed were explored but did not change the magnitude or sign of the association significantly. Calves on dairies that did not test for failure of transfer of passive immunity had an OR of 1.09 (P=0.79) for BRD versus those where they did test. As testing itself is unlikely to influence BRD occurrence in calves, the score is assigned to those dairies not testing or not following up with mitigating actions such as reviewing colostrum testing, feeding or storage.

TABLE 4
Regression coefficients for colostrum management factors for use in a risk
assessment tool for bovine respiratory disease (BRD) in preweaned dairy calves. Regression
coefficients are from analysis of data in a cross-sectional study correlating farm management
practices on 100 California dairies to BRD prevalence in preweaned calves or a longitudinal
study correlating farm management practices on five California dairies to BRD incidence in
preweaned calves.

| | | | Odds | | |
|---|---|---|---|---|---|
| | | | ratio or | | |
| | Regression | Standard | hazard | | |
| Factor | coefficient | error | ratio¹ | p-value | Source² |
| Colostrum source: | | | | | |
| Individual cow | Ref. | | | | |
| Pooled from multiple | 0.54 | 0.54 | 1.72 | 0.31 | BRD 100, UV |
| cows | 0.82 | 0.53 | 2.28 | 0.12 | |
| Mix of sources including | | | | | |
| replacer | | | | | |
| Nursed from dam and | Ref | | | | |
| calved not on pasture | 0.26 | 0.61 | 1.29 | 0.67 | |
| calved on pasture | | | | | |
| Is colostrum supplemented | | | | | |
| with replacer | | | | | |
| No | Ref. | | | | |
| Yes | 0.44 | 0.16 | 1.55 | 0.006 | BRD 10K, UV |
| Is colostrum heat treated? | | | | | |
| Yes | Ref | | | | |
| No | 0.86 | 0.41 | 2.37 | 0.04 | BRD 100, UV |
| If colostrum is heat treated, | | | | | |
| how long is colostrum stored | | | | | |
| before heat treatment | | | | | |
| ≤10 hours | Ref | | | | |
| >10 hours | 1.04 | 0.72 | 2.84 | 0.15 | BRD 100, UV |
| Is colostrum ever tested for | | | | | |
| bacterial content before heat | | | | | |
| treating? | | | | | |
| Yes | Ref | | | | |
| No | 0.47 | 0.41 | 1.60 | 0.25 | BRD 100, UV |
| Is any colostrum from first | | | | | |
| calf heifers used? | | | | | |
| No | Ref | | | | |
| Yes | 0.16 | 0.34 | 1.18 | 0.64 | BRD 100, UV |
| Is a preservative, such as | | | | | |
| potassium sorbate, added to | | | | | |
| colostrum before it is stored? | | | | | |
| No | Ref | | | | |
| Yes | 0.25 | 0.87 | 1.29 | 0.77 | BRD 10K, UV |
| What is the storage container | | | | | |
| for colostrum? | | | | | |
| Bag | Ref | | | | |
| Solid | 0.60 | 0.46 | 1.82 | 0.20 | BRD 100, UV |
| Is colostrum tested for IgG? | | | | | |
| Yes | Ref | | | | |
| No | 1.13 | 0.70 | 3.10 | 0.11 | BRD 10K, UV |
| Are calves fed at least 2.84 L | | | | | |
| (3 quarts) of colostrum in the | | | | | |
| first 12 hours? | Ref | | | | |
| Yes | 0.28 | 0.51 | 1.32 | 0.58 | BRD 100, UV |
| No | | | | | |
| Are calves tested for failure | | | | | |
| of transfer of passive | | | | | |
| immunity? | Ref | | | | |
| Yes | 0.09 | 0.34 | 1.09 | 0.79 | BRD 100, UV |
| No | | | | | |

¹Odds ratio for results from the BRD 100 study, hazard ratio for results from the BRD 10K study; see last column for source
²BRD 100: Study on prevalence of bovine respiratory disease in 4,636 preweaned calves or calves housed in hutches on 100 dairies in California surveyed between May 2014 and April 2016
BRD 10K: Study on incidence of BRD in 11,300 calves followed from birth to weaning on five California dairies between March 2015 and July 2016.
UV = univariable analysis
MV = multivariable analysis

Table 5 shows the variables for milk feeding and the sources of their coefficients. Unless noted in Table 5, all analyses are from univariable analyses in the BRD 100 study. The analysis of feeding order by age resulted in an OR for BRD of 1.90 (P=0.12) for calves on dairies where no feeding order by age existed versus those where youngest were fed first or typically first. On dairies where the oldest calves were fed first or typically first, the OR for BRD in calves versus those calves where the youngest were fed first or typically first was 2.91 (P=0.001). The effect of feeding order by health was only apparent after restricting the sample to those dairies that do not have a hospital pen for sick calves. Feeding order by health status was not significantly associated with BRD in those dairies that had a hospital pen for sick calves, which was found in 10 of the 100 dairies in the data set. Herd size for dairies with hospital pens ranged from 200 to 2,750 lactating cows, they had a mean of 59.1% Holstein cows and 38.3% Jersey cows and 98.7% of calves surveyed on these dairies were housed in individual hutches. Among those dairies that did not have a hospital pen, the odds of BRD were higher in calves where sick calves were fed first or where there was no feeding order in terms of health status (OR 2.36, P=0.03). Feeding youngest or healthy calves first may reduce the risk of disease transmission from sick to healthy. Presumably, if a dairy has a hospital pen for calves, those calves are fed separately from the rest which should improve biocontainment. Disease transmission through fomites, including workers, has been recognized as a risk factor for BRD (Gorden and Plummer, 2010). In a study on the spread of salmonella between calves housed individually, indirect spread by fomites, e.g. buckets or the stockman, was more important than pen design (Hardman P. M., 1991). It is also possible that dairies that elected to feed the youngest and/or healthy calves first tended to be better managed dairies in terms of other BRD risk factors. The scores for milk source are based on univariable analysis from both the BRD 100 and BRD 10K studies. In the BRD 10K study, an HR of 1.77 (P<0.001) for BRD in calves was found for calves being fed milk replacer versus those fed waste milk. Milk replacer feeding has been associated with higher risk of pneumonia in calves compared to feeding waste milk (Godden et al., 2005). The scores for feeding waste milk or a mix of sources are based on univariable analysis from the BRD 100 study, with >90% saleable milk as the reference group. The OR for >90% waste milk versus >90% saleable milk for BRD in calves was 1.58 (P=0.63) while it was 1.85 (P=0.55) for a mix of sources (no source contributing >90%) versus >90% saleable milk. The score for milk pasteurization is from multivariable analysis in the BRD 100 study where calves that did not receive pasteurized milk had an OR of 10.17 (P=0.001) for BRD versus those that did receive pasteurized milk. The benefits of milk pasteurization before feeding to calves are well established (Callan and Garry, 2002). The score for whether milk is tested for bacteria before pasteurization is based on univariable analysis results from the BRD 10K study, where those calves where milk was not tested for bacteria before pasteurization had a HR for BRD of 1.28 (P=0.001) versus those where milk was tested. Both studies produced significant analysis results for the variable describing whether milk is tested after pasteurization (OR 8.51, P=0.04, HR 1.28, P=0.001 for not testing versus testing) and the score is the mean of both regression coefficients. Microbiological testing of milk fed to calves before or after pasteurization is another factor, which in itself does not contribute to BRD in calves without the actions that follow to mitigate issues identified. The score associated with the analysis is therefore assigned for those dairies that do not test and for those that do not follow up with corrective action after bacteria are found in the milk, missing opportunities to reduce BRD risk through review of milking hygiene protocols, or verifying pasteurizer performance. The score for whether milk is supplemented with replacer is from the BRD 10K study where those calves that were fed milk supplemented with replacer had an HR for BRD of 1.97 (P<0.001) versus those where milk was not supplemented with replacer. Supplementation of milk with replacer increased the risk of BRD likely for the same reasons as those that lead to an increase in the odds of BRD in calves fed milk replacer compared to saleable milk, such as higher energy and protein intake or medium-chain fatty acids (Godden et al., 2005) in whole milk. The scores for the amount of milk fed are derived from univariable analysis of BRD 100 data stratified by breed and age, where the volume was assessed for calves more than 21 days old and less than 7 days before weaning, separately for Holstein and Jersey calves. Holstein calves, older than 21 days and younger than 7 days before weaning and fed 3.79 liters (4 quarts) per day had an OR of 15.09 (P<0.001) for BRD versus those fed more. In Jerseys, the OR for BRD among those fed 3.79 liters per day versus those fed more in calves older than 21 days and younger than 7 days before weaning was 4.13 (P=0.049). The analysis was restricted to calves more than 21 days old and stratified by breed, because younger calves and Jerseys were assumed to require less nutrients than older calves and Holsteins. Analysis was restricted to calves that were at least 7 days younger than weaning age to exclude those calves that are being milk restricted as part of the weaning process. In the multivariable analysis of the BRD 100 study, a significant interaction between breed and milk quantity fed was found, with Holstein calves fed less than 2.84 L (3 quarts) per day having increased odds of BRD compared to Jersey calves and Jersey calves fed more than 5.68 L (6 quarts) at decreased odds of BRD compared to Jersey calves fed between 2.84 and 5.68 L. It was chosen to present a stratified univariable analysis here due to easier interpretability. Calves fed once or twice a day versus 3 or more times a day had an OR of 1.89 (P<0.001) for BRD. No studies evaluating feeding frequency on calf health could be found. However, one study found no difference in live weight between calves fed 4.5 L once or 2.25 L twice daily between 10 and 410 days of age (Gleeson and O'Brien, 2012).

TABLE 5
Regression coefficients for milk feeding factors for use in a risk assessment tool for
bovine respiratory disease (BRD) in preweaned dairy calves. Regression coefficients are from
analysis of data in a cross-sectional study correlating farm management practices on 100
California dairies to BRD prevalence in preweaned calves or a longitudinal study correlating
farm management practices on five California dairies to BRD incidence in preweaned calves.

| | | | Odds | | |
|---|---|---|---|---|---|
| | | | ratio or | | |
| | Regression | Standard | hazard | | |
| Factor | coefficient | error | ratio¹ | p-value | Source² |
| Feeding order by age | | | | | |
| Youngest first | Ref | | | | |
| No order | 0.64 | 0.41 | 1.90 | 0.12 | BRD 100, UV |
| Oldest first | 1.07 | 0.32 | 2.91 | 0.001 | |
| Feeding order by health | | | | | |
| If no hospital pen | | | | | |
| Healthy fed first or | Ref | | | | |
| typically fed first | | | | | |
| No order or sick fed first | 0.86 | 0.41 | 2.36 | 0.03 | BRD 100, UV |
| Source of milk ( >90% is:) | | | | | |
| Saleable milk | Ref | | | | |
| Waste milk | 0.46 | 0.96 | 1.58 | 0.63 | BRD 100, UV |
| Milk replacer | 0.57 | 0.05 | 1.77 | <0.001 | BRD 10K UV |
| Mix of sources | 0.62 | 1.02 | 1.85 | 0.55 | BRD 100, UV |
| Is milk pasteurized | | | | | |
| Yes | Ref | | | | |
| No | 2.32 | 0.67 | 10.17 | 0.001 | BRD 100, MV |
| Is milk tested for bacteria before | | | | | |
| pasteurization | | | | | |
| Yes | Ref | | | | |
| No | 0.24 | 0.07 | 1.28 | 0.001 | BRD 10K, UV |
| Is milk tested for bacteria after | | | | | |
| pasteurization | | | | | |
| Yes | Ref | | | | |
| No | 2.14 | 1.04 | 8.51 | 0.04 | BRD 100, UV |
| No | 0.24 | 0.07 | 1.28 | 0.001 | BRD 10K, UV |
| Is milk supplemented with | | | | | |
| replacer? | | | | | |
| No | Ref | | | | |
| Yes | 0.68 | 0.06 | 1.97 | <0.001 | BRD 10K, UV |
| How much milk is fed to calves | | | | | |
| >21 days old and younger than | | | | | |
| 7 days before weaning? | | | | | |
| >4 quarts | Ref | | | | |
| ≤4 quarts | | | | | |
| Holsteins | 2.71 | 0.57 | 15.09 | <0.001 | BRD 100, UV |
| Jerseys | 1.42 | 0.83 | 4.13 | 0.049 | |
| How often is milk fed per day | | | | | |
| 3 or more times | Ref | | | | |
| 1 or 2 times | 0.63 | 0.15 | 1.89 | <0.001 | BRD 100 UV |

¹Odds ratio for results from the BRD 100 study, hazard ratio for results from the BRD 10K study; see last column for source
²BRD 100: Study on prevalence of bovine respiratory disease in 4,636 preweaned calves or calves housed in hutches on 100 dairies in California surveyed between May 2014 and April 2016
BRD 10K: Study on incidence of BRD in 11,300 calves followed from birth to weaning on five California dairies between March 2015 and July 2016.
UV = univariable analysis
MV = multivariable analysis

### Vaccinations

Table 6 shows the variables related to vaccination usage and the sources of their coefficients. Calves on dairies that do not use an intranasal vaccine versus those that did had an OR of 1.43 (P=0.32) for BRD in univariable analysis in the BRD 100 study. The score for injectable vaccines given to calves was based on the univariable analysis from the BRD 10K study, where calves that received no modified live vaccine for respiratory pathogens versus those that did had an HR of 1.21 (P<0.001) for BRD. Protective effects were seen for both killed and modified live vaccines in calves in the two studies (OR 1.68, P=0.67 for BRD in calves receiving no killed vaccine versus those that did in the BRD 100 study). Hence, a distinction is not made between the two vaccine types in the risk assessment and only penalize for not using an injectable vaccine that targets any BRD pathogens. A study comparing unvaccinated calves to calves vaccinated once or twice with a modified live vaccine for viral respiratory pathogens found no difference in BRD risk (Windeyer et al., 2012). The success of vaccinations may depend on multiple factors in the calf including maternal antibodies, timing of vaccinations and pathogens involved in causing BRD. In addition, factors related to the dam such as colostrum management may also impact vaccination success since the volume and timing of feeding the colostrum are important. Calves on dairies where cows received no modified live vaccine for respiratory pathogens versus those where cows did receive such a vaccine had an HR for BRD of 3.07 (P<0.001) in the BRD 10K multivariable analysis. Calves on dairies where cows received no killed vaccine for respiratory pathogens versus those where cows did receive such a vaccine had a HR for BRD of 1.18 (P<0.02) in the BRD 10K multivariable analysis.

TABLE 6
Regression coefficients for vaccine usage for use in a risk assessment tool for bovine
respiratory disease (BRD) in preweaned dairy calves. Regression coefficients are from analysis
of data in a cross-sectional study correlating farm management practices on 100 California
dairies to BRD prevalence in preweaned calves or a longitudinal study correlating farm
management practices on five California dairies to BRD incidence in preweaned calves.

| | | | Odds | | |
|---|---|---|---|---|---|
| | | | ratio or | | |
| | Regression | Standard | hazard | | |
| Factor | coefficient | error | ratio¹ | p-value | Source² |
| Are calves given an | | | | | |
| intranasal vaccine? | | | | | |
| Yes | Ref | | | | |
| No | 0.36 | 0.36 | 1.43 | 0.32 | BRD 100, UV |
| Are calves vaccinated | | | | | |
| with an injectable | | | | | |
| vaccine, either killed or | | | | | |
| modified live? | | | | | |
| Yes | Ref | | | | |
| No | 0.19 | 0.03 | 1.21 | <0.001 | BRD 10K, UV |
| Are cows vaccinated with | | | | | |
| a modified live vaccine? | | | | | |
| Yes | Ref | | | | |
| No | 1.12 | 0.05 | 3.07 | <0.001 | BRD 10K, MV |
| Are cows vaccinated with | | | | | |
| a killed vaccine? | | | | | |
| Yes | Ref | | | | |
| No | 0.12 | 0.05 | 1.18 | 0.02 | BRD 10K, MV |

¹Odds ratio for results from the BRD 100 study, hazard ratio for results from the BRD 10K study; see last column for source
²BRD 100: Study on prevalence of bovine respiratory disease in 4,636 preweaned calves or calves housed in hutches on 100 dairies in California surveyed between May 2014 and April 2016
BRD 10K: Study on incidence of BRD in 11,300 calves followed from birth to weaning on five California dairies between March 2015 and July 2016.
UV = univariable analysis
MV = multivariable analysis

### Housing

Table 7 shows the regression coefficients for housing-related factors and their sources. The first three factors included in the section on housing are related to the role that dust plays with respect to BRD. Airborne pathogen concentration is a function of dust particle density and size, among other factors (Callan and Garry, 2002), and can be a contributing factor in the BRD risk for calves. Calves on dairies where the surface next to the hutches where vehicles traveled was not paved or gravel had an HR of 1.36 (P<0.001) for BRD in univariable analysis in the BRD 10K study. Those calves that were on dairies with dust abatement in place in the form of a water truck, but which was deemed ineffective based on study staff observations, had an HR of 5.31 (P<0.001) for BRD in univariable analysis in the BRD 10K study compared to calves without such measures. During a risk assessment, it is advisable to observe the use of water trucks to see whether they are effective or are contributing to the generation of dust before deciding on a score. In the BRD 100 study, calves on dairies without any dust abatement had an OR of 1.41 (P=0.30) for BRD compared to calves on dairies with dust abatement. Calves on dairies where magnesium chloride was used for dust abatement, had an OR of 0.08 (P=0.007) compared to calves with other or without dust abatement measures in univariable analysis in the BRD 100 study. Whether dust was perceived as a problem was categorized as rarely, weekly or all year round. Calves on dairies where dust was considered a problem all year round had an HR of 1.66 (P=0.001) in multivariable analysis in the BRD 10K. Calves that were housed in groups in the BRD 100 study had an OR of 2.47 (P<0.001) for BRD in univariable analysis compared to calves housed individually. Group-housed calves have a greater risk for respiratory disease than individually housed calves (Curtis et al., 2016) presumably through shared use of a milk supply. The univariable analysis for the risk factor group housing was adjusted for age of calves. Compared to calves that were under an additional roof structure with no side walls, calves that were under no extra roof had an OR for BRD of 13.46 (P=0.001), those under a roof with one to three side walls had an OR for BRD of 9.83 (P=0.008) and those completely indoors had an OR of 3.73 (P=0.22) in multivariable analysis in the BRD 100 study. The protective effect of the additional roof without side walls was attributed to its decrease in heat stress and protection from rain without compromising ventilation. Proper ventilation is an important factor in preventing BRD in calves (Callan and Garry, 2002, Lago et al., 2006, Gorden and Plummer, 2010). Severe respiratory illness was observed in calves experimentally exposed to both cold (5° C.) and warm (35° C.) temperatures 21 days post exposure underlining the effect of both cold and heat stress on BRD in calves. An interaction between calf-to-calf contact and age of calves, in the multivariable BRD 100 study analysis, resulted in an OR of 9.48 (P=0.02) for calves older than 75 days with calf-to-calf contact versus those calves in that age group without contact with other calves. A distance of at least 4 feet between hutches is recommended to prevent calf-to-calf contact and transmission of respiratory pathogens (Callan and Garry, 2002), which was rarely observed on our study dairies. In calves that are close to weaning age, housing or nutrition related factors may become less important and calf-to-calf contact with animals shedding pathogens may become a more important factor for their BRD status. A score for calf-to-calf contact was therefore assigned only to calves in this age group. In multivariable analysis in the BRD 100 study, calves in plastic-walled hutches had an OR of 1.96 (P=0.62) for BRD compared to calves in wooden hutches. The score for calves in metal-walled hutches is a combination of regression coefficients from both the BRD 100 and BRD 10K studies. Even though the result from the longitudinal analysis would have been given preference, regression-coefficients from both studies for metal hutches were averaged, because the hutch type in the BRD 10K study made from wooden walls and metal roof was an unusual hutch type and because the BRD 100 study contained a larger variety of hutch types made from metal. In the BRD 100 study, calves in metal hutches had an OR of 11.19 (P=0.001) for BRD in multivariable analysis compared to calves in hutches with wooden walls. In the BRD 10K study, there was a significant HR of 1.24 (P=0.003) for calves in wooden-walled hutches with a metal roof compared to calves in completely wooden hutches in multivariable analysis. Calves that were housed in hutches made from a mix of materials were combined with group-housed calves in the multivariable analysis in the BRD 100 study and had an OR of 1.42 (P=0.54) for BRD compared to calves housed in individual wooden hutches. Metal hutch walls and roofs may conduct heat more efficiently than wood and result in added heat stress to calves housed in these hutches. Similarly, metal hutches may not insulate calves as well as wooden hutches in the cold season leading to cold stress. There were no studies found in the literature that explore the effect of metal hutch materials on calf health to date. The scores for hutch floors are based on results from univariable analysis in the BRD 100 study, where calves in hutches with plastic covered wire floors had an OR of 17.36 (P=0.02) for BRD compared to calves on wooden floors. This was the case on 7 of 100 dairies in that study. Adjusting for the effect of wall material changed the association by less than 10% (OR 16.71 (P=0.04)). Calves in hutches with concrete, rubber, sand, dirt, grass or bedding-covered floors had an OR of 5.38 (P=0.37) for BRD compared to calves on wooden floors in univariable analysis in the BRD 100 study. Plastic covered wire floors (379 hutches in the current study) are typically not covered by bedding which allows for air flow, however, this may result in a colder hutch microclimate compared to slatted floor or other floor types resulting in cold stress for calves. Furthermore, walls of such hutch types are commonly made of metal (319 of 379 in the current study had either sheet metal, bars or wire metal walls) and may or may not be located in enclosed or semi-enclosed barns (196 of the 379 were in 3 or 4 walled structures) which may further contribute to the increased risk of BRD independent of the floor type. Calves on dairies where manure under the hutches was flushed with recycled lagoon flush water had an OR of 12.06 (P=0.008) for BRD in multivariable analysis in the BRD 100 study compared to calves where no flush system was used for manure. The use of fresh water for flushing away manure was protective in the multivariable BRD 100 model compared to no flush (OR 0.76, P=0.86) and received a score of 0 together with no flush system. The role of recycled lagoon water in BRD risk for preweaned calves could be in the form of noxious fumes such as ammonia, hydrogen sulfide or methane, which may insult the respiratory mucosa and predispose calves to BRD. Calves could also be directly exposed to respiratory pathogens such as BVDV or possibly Coronavirus shed in feces if recycled lagoon water is used to flush feces under hutches.

TABLE 7
Regression coefficients for housing related factors for use in a risk assessment tool for
bovine respiratory disease (BRD) in preweaned dairy calves. Regression coefficients are from
analysis of data in a cross-sectional study correlating farm management practices on 100
California dairies to BRD prevalence in preweaned calves or a longitudinal study correlating
farm management practices on five California dairies to BRD incidence in preweaned calves.

| | Regression | Standard | Odds ratio or | | |
|---|---|---|---|---|---|
| Factor | coefficient | error | hazard ratio¹ | p-value | Source² |
| Are surfaces next to hutches | | | | | |
| paved or gravel? | | | | | |
| Yes | Ref. | | | | |
| No | 0.31 | 0.06 | 1.36 | <0.001 | BRD 10K, UV |
| Method for dust abatement | | | | | |
| None | Ref. | | | | |
| Water truck, not effective | 1.67 | 0.08 | 5.31 | <0.001 | BRD 10K, UV |
| Magnesium chloride | −2.50 | 0.92 | 0.08 | 0.007 | BRD 100, UV |
| None | 0.35 | 0.33 | 1.41 | 0.30 | BRD 100, UV |
| Is dust perceived as a | | | | | |
| problem? | Ref. | | | | |
| Never | 0.50 | 0.15 | 1.66 | 0.001 | BRD 10K, UV |
| Rarely | 0.68 | 0.17 | 1.97 | <0.001 | |
| All year | | | | | |
| Are calves housed in groups? | | | | | |
| No | Ref. | | | | |
| Yes | 0.90 | 0.20 | 2.47 | <0.001 | BRD 100, UV |
| Is there extra cover over the | | | | | |
| hutches? | | | | | |
| Complete roof no side | Ref | | | | |
| walls | 2.60 | 0.80 | 13.46 | 0.001 | BRD 100, MV |
| No cover or roof | 2.29 | 0.87 | 9.83 | 0.008 | |
| Complete roof, 1-3 side | 1.32 | 1.06 | 3.73 | 0.22 | |
| walls | | | | | |
| Completely indoors | | | | | |
| Is there calf-to-calf contact in | | | | | |
| calves older than 75 days? | | | | | |
| No | Ref. | | | | |
| Yes | 2.30 | 0.96 | 9.48 | 0.02 | BRD 100, MV |
| What material are hutches | | | | | |
| made out of? | | | | | |
| Wood | Ref. | | | | |
| Plastic | 0.67 | 1.35 | 1.96 | 0.62 | BRD 100, MV |
| Metal | 0.22 | 0.07 | 1.24 | 0.003 | BRD 10K, MV |
| Metal | 2.42 | 0.71 | 11.19 | 0.001 | BRD100, MV |
| Mix of materials or | 0.35 | 0.57 | 1.42 | 0.54 | BRD100, MV |
| group pens | | | | | |
| What material are hutch | | | | | |
| floors made out of? | | | | | |
| Wood | Ref | | | | |
| Plastic covered wire | 2.85 | 1.17 | 17.36 | 0.02 | BRD 100, UV |
| Other | 1.68 | 1.88 | 5.38 | 0.37 | |
| Is there a flush system for | | | | | |
| manure removal under the | | | | | |
| hutches. | Ref. | | | | |
| None | −0.27 | 1.48 | 0.76 | 0.86 | BRD 100, MV |
| Fresh water | 2.49 | 0.94 | 12.06 | 0.008 | |
| Recycled lagoon water | | | | | |

¹Odds ratio for results from the BRD 100 study, hazard ratio for results from the BRD 10K study; see last column for source
²BRD 100: Study on prevalence of bovine respiratory disease in 4,636 pre-weaned calves or calves housed in hutches on 100 dairies in California surveyed between May 2014 and April 2016
BRD 10K: Study on incidence of BRD in 11,300 calves followed from birth to weaning on five California dairies between March 2015 and July 2016.
UV = univariable analysis
MV = multivariable analysis

### Benchmarking

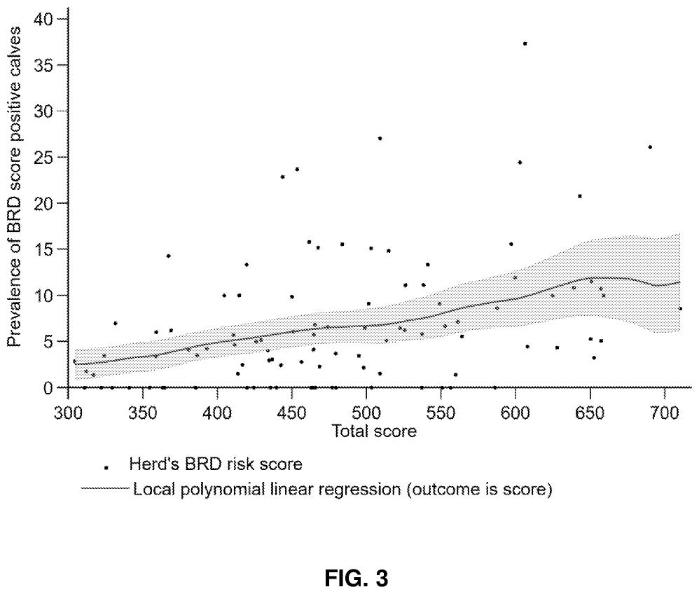

Results for benchmark scores based on the BRD 100 study dairies for each section of the risk assessment and the total score are summarized in Table 8. Despite seven questions not contributing to the benchmarking (due to being follow-up questions or otherwise not applicable) the risk factors are still part of the assessment. The minimum possible score is 0 and the maximum possible score for benchmarking is 4216 prior to being rescaled such that the total score is 1000. The range of scores for the 100 dairies in the BRD 100 study was between 1283 and 2995, the mean score was 2029±42, the median score 1967 (IQR 1746, 2318). FIG. 3 shows how total scaled scores correlate with prevalence in the 100 study dairies of the BRD100 study.

FIG. 3. Scatter plot with smoothed trend line of total scores assigned in a risk assessment for bovine respiratory disease (BRD) in preweaned calves versus prevalence of BRD in a simple random sample of preweaned calves on 100 dairies in California visited between May 2014 and April 2016.

TABLE 8
Benchmark scores developed from regression coefficients for management factors for
use in a risk assessment tool for bovine respiratory disease (BRD) in preweaned dairy calves.
Regression coefficients are from analysis of data in a cross-sectional study correlating farm
management practices on 100 California dairies to BRD prevalence in preweaned calves or a
longitudinal study correlating farm management practices on 5 California dairies to BRD
incidence in preweaned calves. Regression scores were multiplied by 100 and scaled to 1000
for ease of use and interpretation.

| Risk score | 10^{th} | 25^{th} | 50^{th} | 75^{th} | 90^{th} |
|---|---|---|---|---|---|
| section | percentile | percentile | percentile | percentile | percentile |
| Basic Herd | 43 | 47 | 63 | 72 | 81 |
| risk profile¹ | | | | | |
| Maternity pen | 24 | 46 | 65 | 82 | 89 |
| management² | | | | | |
| Colostrum | 41 | 58 | 71 | 82 | 96 |
| management³ | | | | | |
| Milk feeding⁴ | 52 | 79 | 108 | 142 | 155 |
| Vaccinations⁵ | 0 | 3 | 7 | 27 | 31 |
| Calf housing⁶ | 106 | 130 | 170 | 199 | 217 |
| Total risk | 357 | 414 | 467 | 550 | 655 |
| score | | | | | |

¹Herd size, organic status, region, season, herd breed composition; scores ranged between 17 and 103 points
²Bedding changes maternity pen separate for cows and heifers, bedding type, pasture calving, cows and heifers calve together, % calves removed from dam within an hour; scores ranged between 8 and 114 points.
3Source, supplementation with colostrum replacer, heat treatment, storage before heat treatment, testing for bacteria, storage container type, volume fed, testing for failure of transfer of passive immunity; scores ranged between 17 and 105 points.
⁴Feeding order by age, feeding order by health, source, pasteurization, volume fed, feeding frequency; scores ranged between 37 and 230 points.
⁵Intranasal for calves, injectable (killed or modified live) for calves, modified live for cows, killed for cows; scores ranged between 0 and 42 points.
⁶Type of surface next to hutches, dust perceived a problem, group housing, extra shade structures, calf-to-calf contact in calves >75 days, hutch wall material, hutch floor material, flush system for manure; scores ranged between 8 and 263 points.

### Risk Assessment Tool

The complete risk assessment tool included three portions. The first portion requires scoring and totaling each of the six BRD risk management sections (see Table 9 Sections I-VI). Secondly, information is given on how to estimate the preweaned calves' BRD prevalence simultaneous to the risk assessment completion using the California BRD scoring system or the UC Davis BRD application. See Tables 10A-10C. Upon estimating the prevalence, users should document the point prevalence, clinical sign distribution by BRD score status and morbidity (BRD score positive calves) by week of age. Finally, the third portion of the tool aids the user in keeping track of the farm management changes recommended, along with the personnel assigned to implement these changes. See Table 11.

TABLE 9
Sections I-VI. Herd Risk Profile.
I. Basic Herd Risk Profile

| | Risk Factor | Level | Score | Max score | |
|---|---|---|---|---|---|
| 1.1 | What is the average | <250 | 24 | 24 | |
| | number of milking cows | 250-999 | 14 | | |
| | in the herd? | 1000-3999 | 0 | | |
| | | 4000+ | 18 | | |
| 1.2 | Is your dairy a certified | no | 0 | 11 | |
| | organic milk producer | yes | 11 | | |
| 1.3 | What season is it? | Spring (April-June) | 0 | 46 | |
| | | Summer (July-September) | 30 | | |
| | | Fall (October-December) | 46 | | |
| | | Winter (January-March) | 27 | | |
| 1.4 | What percent of the herd | Crossbred | % × 0 = 0 | 22 | |
| | are the following breeds? | Holstein | % × 17 = | | |
| | Use decimals to calculate | Jersey | % × 22 = | | |
| | score, e.g. if | | Total: | | |
| | 60% Holstein and 40% | | | | |
| | Jersey, score is 0.6 × 17 + | | | | |
| | 0.4 × 23 = 10.2 + 9.2 = | | | | |
| | 19.4; round to 19 | | | | |
| | Herd profile risk score | | | 103 | |
| | Your herd's profile contribution to preweaned calves' BRD risk: | | | | |
| | 43 | 47 | 63 | 72 | 81 |
| | Very Low | Low | Moderate | High | Very High |

II. Maternity pen management

| | Risk Factor | Level | Score | Max Score | |
|---|---|---|---|---|---|
| 2.1 | Do cows and heifers | No | 0 | 8 | |
| | calve together? | Yes | 8 | | |
| 2.2 | How often do you | More than 5 times per | 0 | 37 | |
| | change the bedding in | month | | | |
| | the calving pens | 5 or less times per month | | | |
| | | and | | | |
| | | ≥ 95% cows calve | 37 | 37 | |
| | | individually | | | |
| | | ≥ 95% cows calve in | 30 | 30 | |
| | | groups | | | |
| | | Mix of individual | 8 | | |
| | | and group calving | | | |
| | | for cows | | | |
| 2.3. | What type of bedding | Dried manure, gypsum | 0 | 32 | |
| | do you use in the | Sand | 0 | | |
| | calving pens? | Dirt | 32 | | |
| | | Plant materials, i.e. | 12 | | |
| | | shavings, straws, almond | | | |
| | | shells, etc. | | | |
| 2.4. | Do cows or heifers | No and | | 28 | |
| | calve in a pasture? | ≥95% of calves are | 0 | | |
| | | removed from dam | | | |
| | | within an hour | | | |
| | | <95% of calves are | 28 | | |
| | | removed from dam | | | |
| | | within an hour | | | |
| | | Yes and | 0 | | |
| | | ≥95% of calves are | | | |
| | | removed from dam | | | |
| | | within an hour | | | |
| | | <95% of calves are | 23 | | |
| | | removed from dam | | | |
| | | within an hour | | | |
| Maternity pen management risk score | | | | 105 | |
| | Your herd's maternity pen management contribution to preweaned calves' BRD Risk: | | | | |
| | 24 | 46 | 65 | 82 | 89 |
| | Very Low | Low | Moderate | High | Very High |

III. Colostrum management

| | Risk Factor | Level | Score | Max score | |
|---|---|---|---|---|---|
| 3.1. | What is the source | Individual cow | 0 | 19 | |
| | of colostrum? | Pooled | 13 | | |
| | | Mixed sources including | 19 | | |
| | | replacer | | | |
| | | Nursed from dam and | 6 | | |
| | | born on pasture | | | |
| 3.2. | Is colostrum | No | 0 | 10 | |
| | supplemented with | Yes, to improve IgG | 0 | | |
| | replacer? | levels after checking IgG | | | |
| | | Yes, supplemented | 10 | | |
| | | regardless of IgG levels | | | |
| 3.3.a. | Is colostrum heat- | Yes | 0 | 20 | |
| | treated | No | 20 | | |
| 3.3.b. | If colostrum is heat | ≤10 hours | 0 | 25 | |
| | treated, how long is | >10 hours | 25 | | |
| | colostrum stored | | | | |
| | before heat | | | | |
| | treatment | | | | |
| 3.3.c. | Is colostrum ever | Yes | 0 | 11 | |
| | tested for bacterial | No | 11 | | |
| | content before heat | | | | |
| | treating? | | | | |
| 3.4. | Do you use | No | 0 | 4 | |
| | colostrum from | Yes, after ensuring | 0 | | |
| | first calf heifers? | quality is good | | | |
| | | Yes, quality is not | 4 | | |
| | | checked | | | |
| 3.5. | Do you add a | Yes, because we have had | 6 | 6 | |
| | preservative before | issues with colostrum storage | | | |
| | storing colostrum, | in the past | | | |
| | e.g. potassium | Yes, and we haven't had | 0 | | |
| | sorbate? | issues with colostrum storage | | | |
| | | in the past | | | |
| | | No | 0 | | |
| 3.6. | What container is | Bags | 0 | 14 | |
| | colostrum stored | Solid, e.g. bottles | 14 | | |
| | in? | | | | |
| 3.7. | Is colostrum tested | No | 27 | 27 | |
| | for IgG | Yes, and no or very | 0 | | |
| | | little low quality | | | |
| | | colostrum found | | | |
| | | Yes, and when some low | | | |
| | | quality colostrum is | | | |
| | | found we | | | |
| | | supplement or | 0 | | |
| | | discard low quality | | | |
| | | Use anyway | 27 | | |
| 3.8. | Are calves fed at | Yes | 0 | 7 | |
| | least 3 quarts of | No | 7 | | |
| | colostrum in the | | | | |
| | first 12 hours of | | | | |
| | life? | | | | |
| 3.9. | Are calves tested | No | 2 | 2 | |
| | for Transfer of | Yes | | | |
| | Passive Immunity? | And colostrum | 0 | | |
| | | management is | | | |
| | | adjusted if low total | | | |
| | | protein | | | |
| | | And no change in | 2 | | |
| | | management if low | | | |
| | | total protein | | | |
| Colostrum management risk score | | | | 145 | |
| | Your herd's colostrum management contribution to preweaned calves' BRD risk: | | | | |
| | 41 | 58 | 71 | 82 | 96 |
| | Very Low | Low | Moderate | High | Very High |

IV. Milk feeding

| | Risk Factor | Level | Score | Max Score | |
|---|---|---|---|---|---|
| 4.1. | What is the feeding | Youngest first | 0 | 25 | |
| | order for calves by age? | No order by age | 15 | | |
| | | Oldest first | 25 | | |
| 4.2. | What is the feeding | Healthy first or typically first | 0 | 20 | |
| | order by health? | No order or sick fed first | 20 | | |
| 4.3. | What is the source of | Saleable milk | 0 | 15 | |
| | milk that is fed to | Waste milk | 11 | | |
| | calves? | Milk replacer | 14 | | |
| | | Mix of sources | 15 | | |
| 4.4. | Is milk pasteurized? | Yes | 0 | 55 | |
| | | No | 55 | | |
| 4.5. | Is milk tested for | No | 6 | 6 | |
| | bacteria before | Yes and | | | |
| | pasteurization or before | We never find bacteria | 0 | | |
| | feeding? | If bacteria are found, | 0 | 0 | |
| | | protocols are reviewed or | | | |
| | | other action taken | | | |
| | | No particular action taken | 6 | | |
| | | if bacteria are found | | | |
| 4.6. | Is milk ever tested for | No | 28 | 28 | |
| | bacteria after | Yes and | | | |
| | pasteurization, if | Never a problem | 0 | | |
| | applicable? | If bacteria are found, | 0 | | |
| | | protocols are reviewed or | | | |
| | | other action taken | | | |
| | | No particular action taken | 28 | | |
| | | if bacteria are found | | | |
| 4.7. | Is milk supplemented | No | 0 | 16 | |
| | with replacer? | Yes | 16 | | |
| 4.8. | How much milk is fed | >4 quarts per day | 0 | 64 | |
| | per day? | ≤4 quarts per day | | | |
| | If <4 quarts for short | Holstein | 64 | | |
| | period before weaning, | Jersey | 34 | | |
| | don't assign points. | | | | |
| 4.9. | How often is milk fed | 3 or more times per day | 0 | 15 | |
| | per day? | 1 or 2 times per day | 15 | | |
| Calf milk feeding risk score | | | | 244 | |
| | Your herd's calf milk feeding contribution to preweaned calves' BRD risk: | | | | |
| | 52 | 79 | 108 | 142 | 155 |
| | Very Low | Low | Moderate | High | Very High |
| I. Vaccinations | | | | | |
| | Risk Factor | Level | Score | Max Score | |
| 5.1. | Are calves given an | Yes | 0 | 9 | |
| | intranasal vaccine? | No | 9 | | |
| 5.2. | Are calves vaccinated | Yes | 0 | 5 | |
| | with an injectable | No | 5 | | |
| | vaccine, either killed or | | | | |
| | modified live? | | | | |
| 5.3. | Are cows vaccinated with | Yes | 0 | 27 | |
| | a modified live vaccine? | No | 27 | | |
| 5.4. | Are cows vaccinated with | Yes | 0 | 3 | |
| | a killed vaccine? | No | 3 | | |
| Vaccination risk score | | | | 44 | |
| | Your herd's vaccination contribution to preweaned calves' BRD risk: | | | | |
| | 0 | 3 | 7 | 27 | 31 |
| | Very Low | Low | Moderate | High | Very High |
| V. Housing | | | | | |
| | Risk Factor | Level | Score | Max Score | |
| 6.1. | Are surfaces next to | Yes | 0 | 7 | |
| | hutches paved or | No | 7 | | |
| | gravel? | | | | |
| 6.2. | Is there any dust | Water truck | | 40 | |
| | abatement next to the | Effective ( i.e. truck drives | 0 | | |
| | calf hutches? | slowly, does not create | | | |
| | Ideally, observe water | more dust) | | | |
| | truck to judge | Not effective (i.e. truck | 40 | | |
| | effectiveness | produces a lot of dust) | | | |
| | | Magnesium chloride | 0 | | |
| | | None | 8 | | |
| 6.3. | Is dust a problem? | Never | 0 | 16 | |
| | | Rarely | 12 | | |
| | | All year | 16 | | |
| 6.4 | Are calves housed in | No | 0 | 21 | |
| | groups? | Yes | 21 | | |
| 6.5. | Is there extra cover over | Complete roof no side walls | | 62 | |
| | the hutches? | (including shade cloth) | 0 | | |
| | | No cover or roof | 62 | | |
| | | Complete roof, 1-3 side walls | 54 | | |
| | | Completely indoors | 31 | | |
| 6.6. | Do calves older than 75 | No | 0 | 55 | |
| | days have calf-to-calf | Yes | 55 | | |
| | contact? | | | | |
| 6.7. | What material are | Wood | 0 | 31 | |
| | hutch walls made out | Plastic | 16 | | |
| | of? | Metal | 31 | | |
| | | Mix of materials | 8 | | |
| 6.8. | What material are | Wood | 0 | 68 | |
| | hutch floors made out | Plastic coated wire | 68 | | |
| | of? | Other | 40 | | |
| 6.9. | Is there a flush system | None or fresh water | 0 | 59 | |
| | for manure removal | Recycled lagoon water | 59 | | |
| | under the hutches? | | | | |

| | |
|---|---|
| Calf housing risk score | 359 |

Your herd's calf housing contribution to preweaned calves' BRD risk:

| | | | | |
|---|---|---|---|---|
| 106 | 130 | 170 | 199 | 217 |
| Very Low | Low | Moderate | High | Very High |

| BRD risk assessment sections | Your herd's BRD risk score | Maximum score |
|---|---|---|
| I. Herd profile score | | 103 |
| II. Maternity pen management score | | 105 |
| III. Colostrum management score | | 145 |
| IV. Calf milk feeding score | | 244 |
| V. Vaccination score | | 44 |
| VI. Calf housing score | | 359 |
| Total score | | 1000 |

Your herd's preweaned calves' BRD risk:

| | | | | |
|---|---|---|---|---|
| 357 | 414 | 467 | 550 | 655 |
| Very Low | Low | Moderate | High | Very High |

### Tables 10A-10C. Point Prevelance.

Determine the prevalence of BRD in the herd's preweaned calves at the time the risk assessment is conducted using the California BRD scoring system (see FIG. 2), or the UC Davis BRD score smart phone or tablet application (available in English, Spanish and Arabic in iOS and Android systems). Tables 10A-10C below can be completed manually or using the prevalence report feature of the UC Davis BRD score application.

TABLE 10A
I. Point Prevalence

Number of calves in herd
Number of calves scored
Number of score positive
Number of score negative
Prevalence, %

TABLE 10B
II. Breakdown by clinical signs

| Sign | Positive | Percent Positive |
|---|---|---|
| Eye discharge | | |
| Nasal discharge | | |
| Ear droop/head tilt | | |
| Cough | | |
| Abnormal breathing | | |
| Rectal Temperature ≥ 102.5° F. | | |

TABLE 10C
III. Breakdown by age

| Week | Total Positive/Total scored | Percent Positive |
|---|---|---|
| 1 | | |
| 2 | | |
| 3 | | |
| 4 | | |
| 5 | | |
| 6 | | |
| 7 | | |
| 8 | | |
| >8 | | |

TABLE 11
Management Recommendations for Your Farm
Based on the score sheet on the previous pages and a discussion about
feasible changes. A checkmark in a box indicates a recommended
change and follow up are suggested.
I. Maternity pen management

| | | Staff |
|---|---|---|
| | | responsible |
| | | for |
| | | changes/ |
| | Risk factors | Comments |
| [symbol] | Calve cows and heifers separately | |
| [symbol] | Change bedding in cow calving pens more frequently. | |
| | Target 5 or more/month for lower risk scores. | |
| | Aim to change bedding at least: times per month. | |
| | Currently bedding is changed: times per month. | |
| [symbol] | Switch bedding material in calving pen(s) to . | |
| | Currently maternity pens are bedded using | |
| [symbol] | Remove calves more promptly from dams. Goal is to | |
| | remove % of calves within 1 hour of birth from | |
| | the dam. | |
| | Currently % of calves are removed from dam | |
| | within an hour. | |
| [symbol] | Other changes: | |

II. Colostrum management

| | |
|---|---|
| [symbol] | Switch the source of colostrum to individual cows. |
| | Currently you are using . |
| [symbol] | Heat-treat colostrum as recommended. |
| | Currently you are not heat-treating colostrum. |
| [symbol] | Store colostrum fewer than 10 hours before |
| | heat-treating it. |
| | The goal is to store colostrum no more than |
| | before heat treatment. |
| | Currently you are storing colostrum on average for |
| | before heat-treating it. |
| [symbol] | Test colostrum for bacteria before heat treating it. |
| | Currently you are not testing for bacteria. |
| | If you find bacteria in your colostrum, trouble shoot |
| | hygiene for colostrum handling (milking, storage |
| | containers). |
| [symbol] | Check colostrum quality using a colostrometer or |
| | refractometer. If the quality is not sufficient, discard |
| | colostrum or use it to feed older calves. |
| | Currently you are not testing for or discarding low |
| | quality colostrum. |
| [symbol] | Switch to colostrum storage in bags. |
| | Currently you are using to store colostrum. |
| | Feed at least 3 quarts of colostrum to calves in the first |
| | 12 hours of life. |
| | Currently you are feeding |
| [symbol] | Test calves for transfer of passive immunity (FTP) with a |
| | blood sample after 24 hours and before 7 days of life. If |
| | total protein is < 5.2 g/dL, discuss colostrum program |
| | with veterinarian. |
| | Currently you are not testing for FPT. |
| [symbol] | Other |

III. Milk feeding

| | |
|---|---|
| [symbol] | Change the feeding order so that you are always feeding |
| | the youngest calves first. |
| | Currently your milk feeding order by age is: |
| [symbol] | Change the feeding order so that you are feeding the sick |
| | calves last. |
| | Currently your feeding order by health is: |
| [symbol] | Switch the source of the milk you are feeding the calves |
| | to |
| | Currently you are feeding |
| | Pasteurize the milk you are feeding the calves. |
| | Currently you are not pasteurizing the milk fed to calves. |
| [symbol] | Test the milk you are feeding the calves for bacteria |
| | [symbol] Before pasteurization |
| | [symbol] After pasteurization |
| | [symbol] Before feeding (if not pasteurizing) |
| | Currently your protocol for testing the milk is: |
| | If bacteria are found in the milk, take these steps to |
| | troubleshoot |
| | [symbol] Review protocols for storage and feeding hygiene |
| | [symbol] Check pasteurizer for adequate temperature and/or |
| | mixing |
| [symbol] | Feed at least 4 quarts (3.8 liters) of milk per day to all |
| | calves unless they are being conditioned for weaning. |
| | Currently you are feeding per day. |
| [symbol] | Feed calves 3 or more times a day |
| | Currently you are feeding /day |
| [symbol] | Other |

IV. Vaccines

| | |
|---|---|
| [symbol] | Vaccinate calves with an intranasal vaccine for BRD |
| | following manufacturer recommendation |
| [symbol] | Vaccinate calves with an injectable vaccine, either |
| | modified live or killed for BRD following manufacturer |
| | recommendation |
| [symbol] | Vaccinate cows and heifers with a killed vaccine (usually |
| | done at dry-off) following manufacturer recommendation |
| [symbol] | Vaccinate cows and heifers with a modified live vaccine |
| | (usually done at freshening) following manufacturer |
| | recommendation |
| [symbol] | Other |

V. Housing

| | |
|---|---|
| [symbol] | Implement dust abatement |
| | [symbol] Water truck |
| | [symbol] Magnesium chloride |
| | [symbol] Other |
| | Currently the following is done for dust abatement: |
| [symbol] | Install an extra cover over the hutches or calf groups in |
| | the form of |
| | Currently there is no cover over the hutches. |
| [symbol] | Avoid calf-to-calf contact in calves over 75 days old |
| [symbol] | Change hutch type to |
| | Currently calves are housed in hutches made of |
| | Floors: |
| | Walls: |
| | Roofs: |
| [symbol] | Stop using recycled lagoon water to flush under the |
| | hutches. |
| [symbol] | Other |

### Conclusions

The present study offers a comprehensive risk assessment tool for BRD in pre-weaned calves on dairies. Scores are proportional to effect measures observed in data sets including over 11,000 calves on 5 dairies and more than 4,500 calves on 100 dairies. Together with the CA BRD scoring system for pre-weaned calves, the risk assessment tool enables producers, veterinarians and consultants to objectively monitor BRD prevalence and adjust management factors that are dairy specific and that may be the most important contributors to the risk for BRD in their calf herd.

### REFERENCES

California Department of Food and Agriculture, Division of Marketing Services, 2017. California Dairy Statistics 2017.

Callan, R. J. and F. B. Garry. 2002. Biosecurity and bovine respiratory disease. Vet Clin North Am Food Anim Pract 18:57-77. https://doi.org/10.1016/S0749-0720(02)00004-X.

Curtis, G. C., C. M. Argo, D. Jones, and D. H. Grove-White. 2016. Impact of feeding and housing systems on disease incidence in dairy calves. Vet Rec 179(20):512. https://doi.org/10.1136/vr.103895.

Dubrovsky, S. A., A. L. Van Eenennaam, B. M. Karle, P. V. Rossitto, T. W. Lehenbauer, and S. S. Aly. 2019. Epidemiology of bovine respiratory disease (BRD) in preweaned calves on California dairies: The BRD 10K study. J Dairy Sci. 102(8):7306-7319. https://doi.org/10.3168/jds.2018-14774.

Gleeson, D. and B. O'Brien. 2012. Effect of milk feed source, frequency of feeding and age at turnout on calf performance, live-weight at mating and 1st lactation milk production. Ir Vet J 65(1):18. https://doi.org/10.1186/2046-0481-65-18.

Godden, S., R. Bey, K. Lorch, R. Farnsworth, and P. Rapnicki. 2008. Ability of organic and inorganic bedding materials to promote growth of environmental bacteria. J Dairy Sci 91(1):151-159. https://doi.org/10.3168/jds.2007-0415.

Godden, S. M., J. P. Fetrow, J. M. Feirtag, L. R. Green, and S. J. Wells. 2005. Economic analysis of feeding pasteurized nonsaleable milk versus conventional milk replacer to dairy calves. Journal of the American Veterinary Medical Association 226(9):1547-1554.

Godden, S. M., D. M. Haines, K. Konkol, and J. Peterson. 2009. Improving transfer of passive immunoglobulins in calves. II: interaction between feeding method and volume of colostrum fed. J Dairy Sci 92(4):1758-1764. http://dx.doi.org/10.3168/jds.2008-1847.

Gorden, P. J. and P. Plummer. 2010. Control, management, and prevention of bovine respiratory disease in dairy calves and cows. Vet Clin North Am Food Anim Pract 26(2):243-259.

Hardman P. M., W. C. M., Wray C. 1991. Transmission of salmonellae among calves penned individually. The Veterinary Record 129(15):327-329. http://dx.doi.org/10.1016/j.cvfa.2010.03.004.

Holloway, K. L., M. A. Kotowicz, S. E. Lane, S. L. Brennan, and J. A. Pasco. 2015. FRAX (Aus) and falls risk: Association in men and women. Bone 76:1-4. http://dx.doi.org/10.1016/j.bone.2015.03.004.

Kanis, J. A., O. Johnell, A. Oden, H. Johansson, and E. McCloskey. 2008. FRAX and the assessment of fracture probability in men and women from the UK. Osteoporos Int 19(4):385-397.

Lago, A., S. M. McGuirk, T. B. Bennett, N. B. Cook, and K. V. Nordlund. 2006. Calf respiratory disease and pen microenvironments in naturally ventilated calf barns in winter. J Dairy Sci 89(10):4014-4025. http://dx.doi.org/10.1007/s00198-007-0543-5.

Lesnyak, O., S. Sahakyan, A. Zakroyeva, J. P. Bilezikian, N. Hutchings, V. Babalyan, R. Galstyan, A. Lebedev, H. Johansson, N. C. Harvey, E. McCloskey, and J. A. Kanis. 2017. Epidemiology of fractures in Armenia: development of a country-specific FRAX model and comparison to its surrogate. Arch Osteoporos 12(1):98. http://dx.doi.org/10.1007/s11657-017-0392-6.

Love, W. J., T. W. Lehenbauer, B. M. Karle, L. E. Hulbert, R. J. Anderson, A. L. Van Eenennaam, T. B. Farver, and S. S. Aly. 2016a. Survey of management practices related to bovine respiratory disease in preweaned calves on California dairies. J Dairy Sci 99(2):1483-1494. http://dx.doi.org/10.3168/jds.2015-9394.

Love, W. J., T. W. Lehenbauer, P. H. Kass, A. L. Van Eenennaam, and S. S. Aly. 2014. Development of a novel clinical scoring system for on-farm diagnosis of bovine respiratory disease in pre-weaned dairy calves. PeerJ 2:e238. http://dx.doi.org/10.7717/peerj.238.

Love, W. J., T. W. Lehenbauer, A. L. Van Eenennaam, C. M. Drake, P. H. Kass, T. B. Farver, and S. S. Aly. 2016b. Sensitivity and specificity of on-farm scoring systems and nasal culture to detect bovine respiratory disease complex in preweaned dairy calves. J Vet Diagn Invest 28(2):119-128. http://dx.doi.org/10.1177/1040638715626204.

Maier, G. U., W. J. Love, B. M. Karle, S. A. Dubrovsky, D. R. Williams, J. D. Champagne, R. J. Anderson, J. D. Rowe, T. W. Lehenbauer, A. L. Van Eenennaam, and S. S. Aly. 2019. Management factors associated with bovine respiratory disease in preweaned calves on California dairies: The BRD 100 study. J Dairy Sci. 10.3168/jds.2018-14773. http://dx.doi.org/10.3168/jds.2018-14773.

McGuirk, S. M. and M. Collins. 2004. Managing the production, storage, and delivery of colostrum. Vet Clin North Am Food Anim Pract 20(3):593-603. http://dx.doi.org/10.1016/j.cvfa.2004.06.005.

Moons, K. G., F. E. Harrell, and E. W. Steyerberg. 2002. Should scoring rules be based on odds ratios or regression coefficients? J Clin Epidemiol 55(10):1054-1055.

Nandy, S., S. Parsons, C. Cryer, M. Underwood, E. Rashbrook, Y. Carter, S. Eldridge, J. Close, D. Skelton, S. Taylor, G. Feder, and G. Falls Prevention Pilot Steering. 2004. Development and preliminary examination of the predictive validity of the Falls Risk Assessment Tool (FRAT) for use in primary care. J Public Health (Oxf) 26(2):138-143. http://dx.doi.org/10.1093/pubmed/fdh132.

National Action Plan for Combating Antibiotic-Resistant Bacteria. 2015. The White House, Washington, D.C.

Pearson, T. A. 2002. New tools for coronary risk assessment: what are their advantages and limitations? Circulation 105(7):886-892. http://dx.doi.org/10.1161/hc0702.103727.

Povoroznyuk, V. V., N. V. Grygorieva, J. A. Kanis, M. Ev, H. Johansson, N. C. Harvey, M. O. Korzh, S. S. Strafun, V. M. Vaida, F. V. Klymovytsky, R. O. Vlasenko, and V. S. Forosenko. 2017. Epidemiology of hip fracture and the development of FRAX in Ukraine. Arch Osteoporos 12(1):53. http://dx.doi.org/10.1007/s11657-017-0343-2.

Shivley, C. B., J. E. Lombard, N. J. Urie, D. M. Haines, R. Sargent, C. A. Kopral, T. J. Earleywine, J. D. Olson, and F. B. Garry. 2018. Preweaned heifer management on US dairy operations: Part II. Factors associated with colostrum quality and passive transfer status of dairy heifer calves. J Dairy Sci. http://dx.doi.org/10.3168/jds.2017-14008.

Stewart, S., S. Godden, R. Bey, P. Rapnicki, J. Fetrow, R. Farnsworth, M. Scanlon, Y. Arnold, L. Clow, K. Mueller, and C. Ferrouillet. 2005. Preventing bacterial contamination and proliferation during the harvest, storage, and feeding of fresh bovine colostrum. J Dairy Sci 88(7):2571-2578. http://dx.doi.org/10.3168/jds.S0022-0302(05)72933-7.

United States Animal Health Association, 2011. How to Do Risk Assessments and Develop Management Plans for Johne's Disease. 4th ed. Veterinary Services, United States Department of Agriculture.

United States Department of Agriculture 1994. National Dairy Heifer Project 1991-1992, Dairy Heifer Morbidity, Mortality, and Health Management Focusing on Preweaned Heifers. USDA:APHIS:VS, CEAH. Fort Collins, Colo. #129.0294. USDA. 2010. Dairy 2007, Heifer Calf Health and Management Practices on U.S. Dairy Operations. USDA:APHIS: VS, CEAH, Fort Collins, Colo. #550.0110.

United States Department of Agriculture. 2012. Dairy Heifer Raiser, 2011 USDA-APHIS-VS, CEAH, National Animal Health Monitoring System (NAHMS), Fort Collins, Colo. #613.1012.

United States Department of Agriculture. 2016. Dairy 2014, Dairy Cattle Management Practices in the United States, 2014. USDA:APHIS:VS, CEAH. Fort Collins, Colo. #692.0216.

Waltner-Toews, D. S. W. Martin, A. H. Meek. 1986. Dairy calf management, morbidity and mortality in Ontario Holstein herds. III. Association of management with morbidity. Prey Vet Med 4:137-158.

Windeyer, M. C., K. E. Leslie, S. M. Godden, D. C. Hodgins, K. D. Lissemore, and S. J. LeBlanc. 2012. The effects of viral vaccination of dairy heifer calves on the incidence of respiratory disease, mortality, and growth. J Dairy Sci 95(11):6731-6739. http://dx.doi.org/10.3168/jds.2012-5828.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of determining the risk of bovine respiratory disease (BRD) in pre-weaned dairy calves on a dairy farm, comprising:
extracting dairy farm management practice information from a dairy farm, wherein the dairy farm management practice information comprises one or more items of information for two or more dairy farm management practice categories selected from the group consisting of: basic herd risk profile, maternity pen management, colostrum management, milk feeding, vaccinations, and calf housing;
assigning weighted risk scores to the items of dairy farm management practice information extracted; and
determining a total risk score for each of the two or more dairy farm management practice categories based on the assigned weighted risk scores.

2. The method according to claim 1, wherein the dairy farm management practice information comprises one or more items of basic herd risk profile information.

3. The method according to claim 2, wherein the one or more items of basic herd risk profile information comprises one or more of: herd size, breed distribution, organic herd status, and season.

4. The method according to any one of claims 1 to 3, wherein the dairy farm management practice information comprises one or more items of maternity pen management information.

5. The method according to claim 4, wherein the one or more items of maternity pen management information comprises one or more of: frequency of bedding changes in the maternity pen for periparturient cows and heifers, individual or group pen calving for cows and heifers, whether cows and heifers calve together, the materials used for bedding, whether calves are born on pasture, and whether at least 95% of calves are removed from the dam within an hour of birth.

6. The method according to any one of claims 1 to 5, wherein the dairy farm management practice information comprises one or more items of colostrum management information.

7. The method according to claim 6, wherein the one or more items of colostrum management information comprises one or more of: the source of colostrum, whether colostrum was supplemented with replacer, whether the colostrum was heat-treated before feeding, how long the colostrum was stored before heat-treatment, testing for bacterial content of colostrum, use of colostrum from first calf heifers, addition of a preservative, the type of storage container, testing for immunoglobulin content, whether calves received at least 2.84 L (3 quarts) of colostrum in the first 12 hours of life, and whether calves are tested for failure of transfer of passive immunity.

8. The method according to any one of claims 1 to 7, wherein the dairy farm management practice information comprises one or more items of milk feeding information.

9. The method according to claim 6, wherein the one or more items of milk feeding information comprises one or more of: feeding order by age and health of calves, the source of milk, whether the milk is pasteurized, whether the milk is tested for bacteria before or after pasteurization, whether the milk is supplemented with replacer, how much milk is fed each day, and how often milk is fed each day.

10. The method according to any one of claims 1 to 9, wherein the dairy farm management practice information comprises one or more items of vaccination information.

11. The method according to claim 10, wherein the one or more items of vaccination information comprises one or more of: whether vaccines for one or more respiratory pathogens were given, modified live or killed to calves, and modified live or killed to cows.

12. The method according to any one of claims 1 to 11, wherein the dairy farm management practice information comprises one or more items of calf housing information.

13. The method according to claim 12, wherein the one or more items of calf housing information comprises one or more of: the type of driving surfaces next to calf hutches, the presence of dust abatement measures, whether dust was perceived as a problem, whether any pre-weaned calves were housed in groups, the presence of an extra shade structure over the hutches, the presence of calf-to-calf contact in calves>75 days old, hutch wall material, hutch floor material, the presence and type of manure flush system under hutches.

14. The method according to any one of claims 1 to 13, wherein the dairy farm management practice information comprises one or more items of information for three or more dairy farm management practice categories selected from the group consisting of: basic herd risk profile, maternity pen management, colostrum management, milk feeding, vaccinations, and calf housing.

15. The method according to any one of claims 1 to 13, wherein the dairy farm management practice information comprises one or more items of information for four or more dairy farm management practice categories selected from the group consisting of: basic herd risk profile, maternity pen management, colostrum management, milk feeding, vaccinations, and calf housing.

16. The method according to any one of claims 1 to 13, wherein the dairy farm management practice information comprises one or more items of information for five or more dairy farm management practice categories selected from the group consisting of: basic herd risk profile, maternity pen management, colostrum management, milk feeding, vaccinations, and calf housing.

17. The method according to any one of claims 1 to 13, wherein the dairy farm management practice information comprises one or more items of information for each of the following dairy farm management practice categories: basic herd risk profile, maternity pen management, colostrum management, milk feeding, vaccinations, and calf housing.

18. The method according to any one of claims 1 to 17, further comprising determining the risk of BRD in pre-weaned dairy calves on the dairy farm based on the total risk scores for the two or more dairy farm management practice categories.

19. The method according to any one of claims 1 to 18, further comprising recommending to the dairy farm one or more management practice changes based on the extracted dairy farm management practice information.

20. The method according to any one of claims 1 to 19, further comprising producing a report comprising the total risk scores for the two or more dairy farm management practice categories.

21. The method according to claim 20, wherein the report presents the total risk scores on a scale in context with the total risk scores of other dairy farms.

22. The method according to any one of claim 20 or claim 21, wherein the report further comprises the risk of BRD in pre-weaned dairy calves on the dairy farm based on the total risk scores for the two or more dairy farm management practice categories.

23. The method according to claim 22, wherein the report presents the risk of BRD on a scale in context with the risk of BRD of other dairy farms.

24. The method according to any one of claims 1 to 23, wherein the method is computer implemented.

25. The method according to claim 24, wherein the method is implemented using a computer system selected from the group consisting of: a tablet computer, a mobile phone, a laptop computer, and a desktop computer.

26. A non-transitory computer-readable medium comprising instructions which, when executed by a computer system, cause the computer system to:
extract dairy farm management practice information from a dairy farm, wherein the dairy farm management practice information comprises one or more items of information for two or more dairy farm management practice categories selected from the group consisting of: basic herd risk profile, maternity pen management, colostrum management, milk feeding, vaccinations, and calf housing;
assign weighted risk scores to the items of dairy farm management practice information extracted; and
determine a total risk score for each of the two or more dairy farm management practice categories based on the assigned weighted risk scores.

27. The non-transitory computer-readable medium of claim 26, wherein the instructions, when executed by a computer system, cause the computer system to extract the dairy farm management practice information by prompting an individual to input the dairy farm management practice information into the computer system via an input device.

28. The non-transitory computer-readable medium of claim 27, wherein the input device is selected from the group consisting of: a touchscreen display, a mouse, a keyboard, and any combination thereof.

29. The non-transitory computer-readable medium of any one of claims 26 to 28, wherein the instructions, when executed by a computer system, further cause the computer system to:
determine the risk of BRD in pre-weaned dairy calves on the dairy farm based on the total risk scores for the two or more dairy farm management practice categories.

30. The non-transitory computer-readable medium of any one of claims 26 to 29, wherein the instructions, when executed by a computer system, further cause the computer system to: recommend to the dairy farm one or more management practice changes based on the extracted dairy farm management practice information.

31. The non-transitory computer-readable medium of any one of claims 26 to 30, wherein the instructions, when executed by a computer system, further cause the computer system to:
produce a report comprising the total risk scores for the two or more dairy farm management practice categories.

32. The non-transitory computer-readable medium of claim 31, wherein the report presents the total risk scores on a scale in context with the total risk scores of other dairy farms.

33. The non-transitory computer-readable medium of claim 31 or claim 32, wherein the report further comprises the risk of BRD in dairy calves on the dairy farm based on the total risk scores for the two or more dairy farm management practice categories.

34. The non-transitory computer-readable medium of claim 33, wherein the report presents the risk of BRD on a scale in context with the risk of BRD of other dairy farms.

35. The non-transitory computer-readable medium of any one of claims 26 to 34, wherein the non-transitory computer-readable medium is present on a local computer device.

36. The non-transitory computer-readable medium of any one of claims 26 to 34, wherein the non-transitory computer-readable medium is present on a remote server.

37. A computer device comprising:
one or more processors; and
the non-transitory computer readable medium of any one of claims 26 to 34.

38. The computer device of claim 37, wherein the computer device is selected from the group consisting of: a tablet computer, a smartphone, a laptop computer, a desktop computer, and a remote server.
